SEQUENCE LISTING (21 sequences)

SEQ ID NO: 1 - protein, 4 aa, Artificial Sequence
  completely synthetic sequence
CVLS

SEQ ID NO: 2 - protein, 15 aa, Artificial Sequence
  completely synthetic sequence
GKKKKKKSKTKCVIM

SEQ ID NO: 3 - DNA, 52 bp, Artificial Sequence
  completely synthetic sequence
gagagggaattcgggcccttcctgcatgctgctgctgctgctgctgctgggc

SEQ ID NO: 4 - DNA, 41 bp, Artificial Sequence
  completely synthetic sequence
gagagagctcgaggttaacccgggtgcgcggcgtcggtggt

SEQ ID NO: 5 - DNA, 42 bp, Artificial Sequence
  completely synthetic sequence
gagagagtctagagttaacccgtggtccccgcgttgcttcct

SEQ ID NO: 6 - DNA, 43 bp, Artificial Sequence
  completely synthetic sequence
gaagaggaagcttggtaccgccactgggctgtaggtggtggct

SEQ ID NO: 7 - DNA, 27 bp, Artificial Sequence
  completely synthetic sequence
ggcagagctcgtttagtgaaccgtcag

SEQ ID NO: 8 - DNA, 27 bp, Artificial Sequence
  completely synthetic sequence
gagagatctcaaggacggtgactgcag

SEQ ID NO: 9 - DNA, 86 bp, Artificial Sequence
  completely synthetic sequence
tctcctcgaggccaccatggggagtagcaagagcaagcctaaggaccccagccagcgccggatgacagaatacaagcttgtggtgg

SEQ ID NO: 10 - DNA, 33 bp, Artificial Sequence
  completely synthetic sequence
cacatctagatcaggacagcacagacttgcagc

SEQ ID NO: 11 - DNA, 41 bp, Artificial Sequence
  completely synthetic sequence
tctcctcgaggccaccatgacagaatacaagcttgtggtgg

SEQ ID NO: 12 - DNA, 38 bp, Artificial Sequence
  completely synthetic sequence
cactctagactggtgtcagagcagcacacacttgcagc

SEQ ID NO: 13 - DNA, 38 bp, Artificial Sequence
  completely synthetic sequence
gagagaattcgccaccatgacggaatataagctggtgg

SEQ ID NO: 14 - DNA, 33 bp, Artificial Sequence
  completely synthetic sequence
gagagtcgacgcgtcaggagagcacacacttgc

SEQ ID NO: 15 - DNA, 22 bp, Artificial Sequence
  completely synthetic sequence
ccgccggcctggaggagtacag

SEQ ID NO: 16 - DNA, 38 bp, Artificial Sequence
  completely synthetic sequence
gagagaattcgccaccatgactgagtacaaactggtgg

SEQ ID NO: 17 - DNA, 32 bp, Artificial Sequence
  completely synthetic sequence
gagagtcgacttgttacatcaccacacatggc

SEQ ID NO: 18 - DNA, 21 bp, Artificial Sequence
  completely synthetic sequence
gttggagcagttggtgttggg

SEQ ID NO: 19 - DNA, 38 bp, Artificial Sequence
  completely synthetic sequence
gagaggtaccgccaccatgactgaatataaacttgtgg

SEQ ID NO: 20 - DNA, 36 bp, Artificial Sequence
  completely synthetic sequence
ctctgtcgacgtatttacataattacacactttgtc

SEQ ID NO: 21 - DNA, 24 bp, Artificial Sequence
  completely synthetic sequence
gtagttggagctgttggcgtaggc

(19) United States
(12) Patent Application Publication
(10) Pub. No.: US 2002/0193283 A1
(43) Pub. Date: Dec. 19, 2002

(54) Inhibitors of prenyl-protein transferase

(76) Inventors: Christopher J. Dinsmore, Schwenksville, PA (US); Jeffrey M. Bergman, Perkasie, PA (US)

Correspondence Address: MERCK AND CO INC, P O BOX 2000, RAHWAY, NJ, 070650907

(21) Appl. No.: 09/784,818

(22) Filed: Feb. 16, 2001

Related U.S. Application Data
(60) Provisional application No. 60/183,451, filed on Feb. 18, 2000.

(51) Int. Cl. A61K 31/00

(52) U.S. Cl. 514/1

(57) ABSTRACT

The present invention comprises piperazine-containing compounds which inhibit prenyl-protein transferases, including farnesyl-protein transferase and geranylgeranyl-protein transferase type I. Such therapeutic compounds are useful in the treatment of cancer.

### BACKGROUND OF THE INVENTION

The present invention relates to certain compounds that are useful for the inhibition of prenyl-protein transferases and the treatment of cancer. In particular, the invention relates to prenyl-protein transferase inhibitors which are efficacious in vivo as inhibitors of geranylgeranyl-protein transferase type I (GGTase-I) and that inhibit the cellular processing of both the H-Ras protein and the K4B-Ras protein.

Prenylation of proteins by prenyl-protein transferases represents a class of post-translational modification (Glomset, J. A., Gelb, M. H., and Farnsworth, C. C. (1990). Trends Biochem. Sci. 15, 139-142; Maltese, W. A. (1990). FASEB J. 4, 3319-3328). This modification typically is required for the membrane localization and function of these proteins. Prenylated proteins share characteristic C-terminal sequences including CAAX (C, Cys; A, an aliphatic amino acid; X, another amino acid), XXCC, or XCXC. Three post-translational processing steps have been described for proteins having a C-terminal CAAX sequence: addition of either a 15 carbon (farnesyl) or 20 carbon (geranylgeranyl) isoprenoid to the Cys residue, proteolytic cleavage of the last 3 amino acids, and methylation of the new C-terminal carboxylate (Cox, A. D. and Der, C. J. (1992a). Critical Rev. Oncogenesis 3:365-400; Newman, C. M. H. and Magee, A. I. (1993). Biochim. Biophys. Acta 1155:79-96). Some proteins may also have a fourth modification: palmitoylation of one or two Cys residues N-terminal to the farnesylated Cys. While some mammalian cell proteins terminating in XCXC are carboxymethylated, it is not clear whether carboxy methylation follows prenylation of proteins terminating with a XXCC motif (Clarke, S. (1992). Annu. Rev. Biochem. 61, 355-386). For all of the prenylated proteins, addition of the isoprenoid is the first step and is required for the subsequent steps (Cox, A. D. and Der, C. J. (1992a). Critical Rev. Oncogenesis 3:365-400; Cox, A. D. and Der, C. J. (1992b) Current Opinion Cell Biol. 4:1008-1016).

Three enzymes have been described that catalyze protein prenylation: farnesyl-protein transferase (FPTase), geranylgeranyl-protein transferase type I (GGPTase-I), and geranylgeranyl-protein transferase type-II (GGPTase-II, also called Rab GGPTase). These enzymes are found in both yeast and mammalian cells (Clarke, 1992; Schafer, W. R. and Rine, J. (1992) Annu. Rev. Genet. 30:209-237). Each of these enzymes selectively uses farnesyl diphosphate or geranyl-geranyl diphosphate as the isoprenoid donor and selectively recognizes the protein substrate. FPTase farnesylates CaaX-containing proteins that end with Ser, Met, Cys, Gln or Ala. For FPTase, CaaX tetrapeptides comprise the minimum region required for interaction of the protein substrate with the enzyme. The enzymological characterization of these three enzymes has demonstrated that it is possible to selectively inhibit one with little inhibitory effect on the others (Moores, S. L., Schaber, M. D., Mosser, S. D., Rands, E., O'Hara, M. B., Garsky, V. M., Marshall, M. S., Pompliano, D. L., and Gibbs, J. B., J. Biol. Chem., 266:17438 (1991), U.S. Pat. No. 5,470,832).

The prenylation reactions have been shown genetically to be essential for the function of a variety of proteins (Clarke, 1992; Cox and Der, 1992a; Gibbs, J. B. (1991). Cell 65: 1-4; Newman and Magee, 1993; Schafer and Rine, 1992). This requirement often is demonstrated by mutating the CaaX Cys acceptors so that the proteins can no longer be prenylated. The resulting proteins are devoid of their central biological activity. These studies provide a genetic “proof of principle” indicating that inhibitors of prenylation can alter the physiological responses regulated by prenylated proteins.

The Ras protein is part of a signaling pathway that links cell surface growth factor receptors to nuclear signals initiating cellular proliferation. Biological and biochemical studies of Ras action indicate that Ras functions like a G-regulatory protein. In the inactive state, Ras is bound to GDP. Upon growth factor receptor activation, Ras is induced to exchange GDP for GTP and undergoes a conformational change. The GTP-bound form of Ras propagates the growth stimulatory signal until the signal is terminated by the intrinsic GTPase activity of Ras, which returns the protein to its inactive GDP bound form (D. R. Lowy and D. M. Willumsen, Ann. Rev. Biochem. 62:851-891 (1993)). Activation of Ras leads to activation of multiple intracellular signal transduction pathways, including the MAP Kinase pathway and the Rho/Rac pathway (Joneson et al., Science 271:810-812).

Mutated ras genes are found in many human cancers, including colorectal carcinoma, exocrine pancreatic carcinoma, and myeloid leukemias. The protein products of these genes are defective in their GTPase activity and constitutively transmit a growth stimulatory signal.

The Ras protein is one of several proteins that are known to Undergo post-translational modification. Farnesyl-protein transferase utilizes farnesyl pyrophosphate to covalently modify the Cys thiol group of the Ras CAAX box with a farnesyl group (Reiss et al., Cell, 62:81-88 (1990); Schaber et al., J. Biol. Chem., 265:14701-14704 (1990); Schafer et al., Science, 249:1133-1139 (1990); Manne et al., Proc. Natl. Acad. Sci USA, 87:7541-7545 (1990)).

Ras must be localized to the plasma membrane for both normal and oncogenic functions. At least 3 post-translational modifications are involved with Ras membrane localization, and all 3 modifications occur at the C-terminus of Ras. The Ras C-terminus contains a sequence motif termed a “CAAX” or “Cys-Aaa¹-Aaa²-Xaa” box (Cys is cysteine, Aaa is an aliphatic amino acid, the Xaa is any amino acid) (Willumsen et al., Nature 310:583-586 (1984)). Depending on the specific sequence, this motif serves as a signal sequence for the enzymes farnesyl-protein transferase or geranylgeranyl-protein transferase, which catalyze the alkylation of the cysteine residue of the CAAX motif with a C₁₅ or C₂₀ isoprenoid, respectively. (S. Clarke., Ann. Rev. Biochem. 61:355-386 (1992); W. R. Schafer and J. Rine, Ann. Rev. Genetics 30:209-237 (1992)). Direct inhibition of farnesyl-protein transferase would be more specific and attended by fewer side effects than would occur with the required dose of a general inhibitor of isoprene biosynthesis.

Other farnesylated proteins include the Ras-related GTP-binding proteins such as RhoB, fungal mating factors, the nuclear lamins, and the gamma subunit of transducin. James, et al., J. Biol. Chem. 269, 14182 (1994) have identified a peroxisome associated protein Pxf which is also farnesylated. James, et al., have also suggested that there are farnesylated proteins of unknown structure and function in addition to those listed above.

Inhibitors of farnesyl-protein transferase (FPTase) have been described in two general classes. The first class includes analogs of farnesyl diphosphate (FPP), while the second is related to protein substrates (e.g., Ras) for the enzyme. The peptide derived inhibitors that have been described are generally cysteine containing molecules that are related to the CAAX motif that is the signal for protein prenylation. (Schaber et al., ibid; Reiss et. al., ibid; Reiss et al., PNAS, 88:732-736 (1991)). Such inhibitors may inhibit protein prenylation while serving as alternate substrates for the farnesyl-protein transferase enzyme, or may be purely competitive inhibitors (U.S. Pat. No. 5,141,851, University of Texas; N. E. Kohl et al., Science, 260:1934-1937 (1993); Graham, et al., J. Med. Chem., 37, 725 (1994)).

Mammalian cells express four types of Ras proteins (H-, N-, K4A-, and K4B-Ras) among which K4B-Ras is the most frequently mutated form of Ras in human cancers. The genes that encode these proteins are abbreviated H-ras, N-ras, K4A-ras and K4B-ras respectively. H-ras is an abbreviation for Harvey-ras. K4A-ras and K4B-ras are abbreviations for the Kirsten splice variants of ras that contain the 4A and 4B exons, respectively. Inhibition of farnesyl-protein transferase has been shown to block the growth of H-ras-transformed cells in soft agar and to modify other aspects of their transformed phenotype. It has also been demonstrated that certain inhibitors of farnesyl-protein transferase selectively block the processing of the H-Ras oncoprotein intracellularly (N. E. Kohl et al., Science, 260:1934-1937 (1993) and G. L. James et al., Science, 260:1937-1942 (1993). Recently, it has been shown that an inhibitor of farnesyl-protein transferase blocks the growth of H-ras-dependent tumors in nude mice (N. E. Kohl et al., Proc. Natl. Acad. Sci U.S.A., 91:9141-9145 (1994) and induces regression of mammary and salivary carcinomas in H-ras transgenic mice (N. E. Kohl et al., Nature Medicine, 1:792-797 (1995).

Indirect inhibition of farnesyl-protein transferase in vivo has been demonstrated with lovastatin (Merck & Co., Rahway, N.J.) and compactin (Hancock et al., ibid; Casey et al., ibid; Schafer et al., Science 245:379 (1989)). These drugs inhibit HMG-CoA reductase, the rate limiting enzyme for the production of poly-isoprenoids including farnesyl pyrophosphate. Inhibition of farnesyl pyrophosphate biosynthesis by inhibiting HMG-CoA reductase blocks Ras membrane localization in cultured cells.

It has been disclosed that the lysine-rich region and terminal CVIM sequence of the C-terminus of K-RasB confer resistance to inhibition of the cellular processing of that protein by certain selective FPTase inhibitors. (James, et al., J. Biol. Chem. 270, 6221 (1995)) Those FPTase inhibitors were effective in inhibiting the processing of H-Ras proteins. James et al., suggested that prenylation of the K4B-Ras protein by GGTase-I contributed to the resistance to the selective FPTase inhibitors.

Selective inhibitors of GGTase-I have been previously disclosed (see for example U.S. Pat. No. 5,470,832, issued Nov. 28, 1995). Other compounds have been described as selective inhibitors of GGTase-I (see for example PCT Publication No. WO 96/21456). Combinations of a selective inhibitor of FPTase and a selective inhibitor of GGTase-I have been disclosed as useful in the treatment of cancer (PCT Publication No. WO 97/34664).

Several groups of scientists have recently disclosed compounds that are non-selective FPTase/GGTase-I inhibitors. (Nagasuet al. Cancer Research, 55:5310-5314 (1995); PCT application WO 95/25086).

It is the object of the instant invention to provide a prenyl-protein transferase inhibitor which is efficacious in vivo as an inhibitor of geranylgeranyl-protein transferase type I (GGTase-1), also known as CAAX GGTase.

It is also the object of the present invention to provide a compound which inhibits the cellular processing of both the H-Ras protein and the K4B-Ras protein.

It is also the object of the present invention to provide a compound which is efficacious in vivo as an inhibitor of the growth of cancer cells characterized by a mutated K4B-Ras protein.

A composition which comprises such an inhibitor compound is used in the present invention to treat cancer.

### SUMMARY OF THE INVENTION

The present invention comprises piperazine-containing compounds which inhibit prenyl-protein transferases. Further contained in this invention are chemotherapeutic compositions containing these prenyl transferase inhibitors and methods for their production.

The compounds of this invention are illustrated by the formula A:

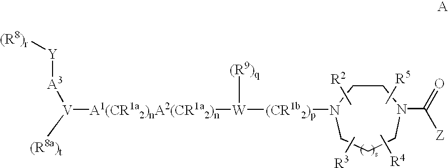

### DETAILED DESCRIPTION OF THE INVENTION

The compounds of this invention are useful in the inhibition of prenyl-protein transferases and the prenylation of the oncogene protein Ras. In a first embodiment of this invention, the inhibitors of prenyl-protein transferases are illustrated by the formula A:

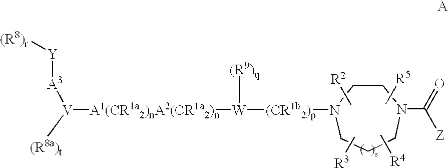

wherein:

R^{1a} and R^{1b} are independently selected from the group consisting of:

a) hydrogen,

b) aryl,

c) heterocyclyl,

d) C₃-C₁₀ cycloalkyl,

e) C₂-C₆ alkenyl,

f) C₂-C₆ alkynyl,

g) R¹⁰O—,

h) R¹⁰S(O)ₘ—,

i) R¹⁰C(O)NR¹⁰—,

j) (R¹⁰)₂NC(O)—,

k) CN,

l) halo,

m) R¹⁰C(O)—,

n) R¹⁰C(O)—,

o) —N(R¹⁰)₂,

p) R¹¹OC(O)NR¹⁰—, and

q) C₁-C₆ alkyl, said alkyl optionally substituted with aryl, heterocyclyl, C₃-C₁₀ cycloalkyl, C₂-C₆ alkenyl, C₂-C₆ alkynyl, R¹⁰O—, R¹¹S(O)ₘ—, R¹⁰C(O)NR¹⁰—, (R¹⁰)₂NC(O)—, CN, halo, R¹⁰C(O)—, R¹⁰C(O)—, —N(R¹⁰)₂, or R¹¹OC(O)—NR¹⁰—;

R² and R³ are independently selected from the group consisting of:

a) H,

b) C₁₋₈ alkyl,

c) C₂₋₈ alkenyl,

d) C₂₋₈ alkynyl,

e) aryl,

f) heterocyclyl,

g) (C═O)NR⁶R⁷, and

h) (C═O)OR⁶,

said alkyl, alkenyl, alkynyl, aryl, and heterocyclyl optionally substituted with one or more substituents selected from the group consisting of:

1) aryl or heterocyclyl, unsubstituted or substituted with:

a) C₁₋₄ alkyl,

b) (CH₂)ₚOR⁶,

c) (CH₂)ₚNR⁶R⁷,

d) halo,

e) CN,

2) C₃₋₆ cycloalkyl,

3) OR⁶,

4) SOₘR^{6a},

5) NR⁶R⁷,

6) NR⁶(C═O)R⁷,

7) NR⁶(C═O)NR⁷R^{7a},

8) —O(C═O)NR⁶R⁷,

9) O(C═O)OR⁶,

10) —(C═O)NR⁶R⁷,

11) —SO₂NR⁶R⁷,

12) NR⁶SO₂R^{6a},

13) —(C═O)R⁶,

14) —(C═O)OR⁶, and

15) halo, or

R² and R³ are attached to the same C atom and are combined to form —(CH₂)ᵤ— wherein one of the carbon atoms is optionally replaced by a moiety selected from: O, S(O)ₘ, —NC(O)—, and —N(COR¹⁰)—;

R⁴ and R⁵ are independently selected from H and C₁₋₄ alkyl;

R⁶, R⁷ and R^{7a} are independently selected from the group consisting of:

a) H,

b) C₁₋₈ alkyl,

c) C₃₋₆ cycloalkyl,

d) heterocyclyl,

e) aryl,

f) aroyl,

g) heteroaroyl,

h) arylsulfonyl, and

i) heteroarylsulfonyl,

said alkyl, cycloalkyl, heterocyclyl, aryl, aroyl, heteroaroyl, arylsulfonyl, and heteroarylsulfonyl is optionally substituted with one or more of the following:

1) C₁₋₄ alkoxy,

2) aryl,

3) heterocyclyl,

4) halo,

5) OH,

6) —(C═O)R¹¹,

7) —SO₂R¹¹,

8) C₁₋₄ alkyl, or

9) N(R¹⁰)₂;

R⁶ and R⁷ may be joined in a ring;

R⁷ and R⁷a may be joined in a ring;

R^{6a} is selected from the group consisting of:

a) C₁₋₄ alkyl,

b) C₃₋₆ cycloalkyl,

c) heterocyclyl, and

d) aryl,

said alkyl, cycloalkyl, heterocyclyl, and aryl is optionally substituted with one or more of the following:

1) C₁₋₄ alkoxy,

2) aryl,

3) heterocyclyl,

4) halogen,

5) OH,

6) —(C═O)R¹¹,

7) —SO₂R¹¹,

8) C₁₋₄ alkyl, or

9) N(R¹⁰)₂;

R⁸ is selected from the group consisting of:

a) aryl,

b) heterocyclyl,

c) C₃-CIO cycloalkyl,

d) C₂-C₆ alkenyl,

e) C₂-C₆ alkynyl,

f) C₁-C₆ perfluoroalkyl,

g) halo,

h) R¹⁰O—,

i) R¹¹S(O)ₘ—,

j) R¹¹C(O)NR¹⁰—,

k) (R¹⁰)₂NC(O)—,

l) CN,

m) R¹⁰C(O)—,

n) R¹⁰OC(O)—,

o) —N(R¹⁰)₂,

p) R¹¹OC(O)NR¹⁰—, and

q) C₁-C₆ alkyl, said alkyl is optionally substituted with aryl, cyanophenyl, heterocyclyl, C₃-C₁₀ cycloalkyl, C₂-C₆ alkenyl, C₂-C₆ alkynyl, C₁-C₆ perfluoroalkyl, halo, R¹⁰O—, R¹¹S(O)ₘ—, R¹⁰C(O)NR¹⁰—, (R¹⁰)₂ NC(O)—, CN, R¹⁰C(O)—, R¹⁰OC(O)—, —N(R¹⁰)₂, or R¹¹OC(O)N R¹⁰—;

R8a is selected from the group consisting of:

a) aryl,

b) heterocyclyl,

c) C₃-C₁₀ cycloalkyl,

d) C₂-C₆ alkenyl,

e) C₂-C₆ alkynyl,

f) C₁-C₆ perfluoroalkyl,

g) halo,

h) R¹⁰O—,

i) R¹¹S(O)ₘ—,

j) R¹⁰C(O)NR¹⁰—,

k) (R¹⁰)2NC(O)—,

l) CN,

m) R¹⁰C(O)—,

n) R¹⁰OC(O)—,

o) —N(R¹⁰)₂,

p) R¹¹OC(O)NR¹⁰—, and

q) C₁-C₆ alkyl unsubstituted or substituted by aryl, cyanophenyl, heterocycle, C₃-C₁₀ cycloalkyl, C₂-C₆ alkenyl, C₂-C₆ alkynyl, C₁-C₆ perfluoroalkyl, halo, R¹⁰O—, R¹¹S(O)ₘ—, R¹⁰C(O)NR¹⁰—, (R¹⁰)₂ NC(O)—, CN, R¹⁰C(O)—, R¹⁰OC(O)—, —N(R¹⁰)₂, or R¹¹ OC(O)NR¹⁰—;

R⁹ is selected from the group consisting of:

a) hydrogen,

b) C₂-C₆ alkenyl,

c) C₂-C₆ alkynyl,

d) C₁-C₆ perfluoroalkyl,

e) halo,

f) R¹⁰O—,

g) R¹¹S(O)ₘ—,

h) R¹⁰C(O)NR¹⁰—,

i) (R¹⁰)₂NC(O)—,

j) CN,

k) R¹⁰C(O)—,

l) R¹⁰OC(O)—,

m) —N(R¹⁰)₂,

n) R¹¹OC(O)NR¹⁰—, and

o) C₁-C₆ alkyl, said alkyl is optionally substituted with perfluoroalkyl, halo, R¹⁰O—, R¹¹S(O)ₘ—, R¹⁰C(O)NR¹⁰—, (R¹⁰)₂NC(O)—, CN, R¹⁰C(O)—, R¹⁰OC(O)—, —N(R¹⁰)₂, or R¹¹OC(O)NR¹⁰—;

R¹⁰ is hydrogen, C₁-C₈ alkyl, C₁-C₆ perfluoroalkyl, benzyl or aryl, said alkyl optionally substituted with OH or —OC₁-C₆ alkyl;

R¹¹ is C₁-C₆ alkyl or aryl;

A¹ and A² are independently selected from the group consisting of

a) a bond,

b) —CH═CH—,

c) —C≡C—,

d) —C(O)—,

e) —C(O)NR¹⁰—,

f) —NR¹⁰C(O)—,

g) —O—,

h) —N(R¹⁰)—,

i) —S(O)₂N(R¹⁰)—,

j) —N(R¹⁰)S(O)₂—, and

k) —S(O)ₘ—;

A³ is —C(O)—, —C(R^{1a})₂—, —O—, —N(R¹⁰)— or —S(O)M—;

V is heteroaryl or aryl;

W is heterocyclyl;

Y is aryl;

Z is aryl or heterocyclyl,

said aryl and heterocyclyl is optionally substituted with one or more of the following:

1) C₁₋₈ alkyl, said alkyl optionally substituted with:

a) C₁₋₄ alkoxy,

b) NR⁶R⁷,

c) C₃₋₆ cycloalkyl,

d) aryl,

e) heterocyclyl,

f) OH,

g) —S(O)ₘR^{6a}, or

h) —C(O)NR⁶R⁷,

2) aryl,

3) heterocyclyl,

3) halo,

4) OR⁶,

5) NR⁶R⁷,

6) CN,

7) CF₃,

9) —S(O)mR^{6a},

10) —C(O)NR⁶R⁷, and

11) C₃-C₆ cycloalkyl;

m is 0, 1 or 2;

n is 0,1,2,3or 4;

p is 0, 1,2,3or 4;

q is 1 or 2;

r is 0, 1,2,3,4,or 5;

s is 0 or 1;

t is 0, 1,2,3,4or 5; and

u is 4or 5;

or a pharmaceutically acceptable salt, stereoisomer or mixture thereof.

Another embodiment of the invention is illustrated by the compounds of Formula B:

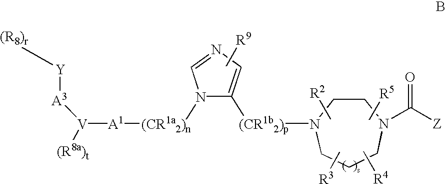

wherein:

R^{1a} and R^{1b} are independently hydrogen or C₁-C₆ alkyl, said alkyl optionally substituted with aryl, C₃-C₁₀ cycloalkyl, halo, R¹⁰O— or —N(R¹⁰)₂;

R², R³, R⁴ and R⁵ are independently selected from H and C₁₋₄ alkyl;

R⁶ and R⁷ are independently selected from the group consisting of:

a) H,

b) C₁₋₈ alkyl,

c) C₃₋₆ cycloalkyl,

d) aryl, and

e) heterocyclyl,

said alkyl, cycloalkyl, aryl, and heterocyclyl optionally substituted with:

1) C₁₋₄ alkoxy,

2) halo,

3) aryl,

4) heterocyclyl, or

5) C₁₋₄alkyl;

R^{6a} is selected from:

a) C₁₋₄ alky,

b) C₃₋₆ cycloalkyl,

c) aryl, and

d) heterocyclyl,

said alkyl, cycloalkyl, aryl, and heterocyclyl optionally substituted with:

1) C₁₋₄ alkoxy,

2) halo,

3) aryl,

4) heterocyclyl, or

5) C₁₋₄alkyl;

R⁸ is independently selected from the group consisting of:

a) aryl,

b) C₂-C₆ alkenyl,

c) C₂-C₆ alkynyl,

d) C₁-C₆ perfluoroalkyl,

e) halo,

f) R¹⁰O—,

g) R¹⁰C(O)NR¹⁰—,

h) CN,

i) R¹⁰C(O)—,

j) R¹⁰OC(O)—,

k) —N(R¹⁰)₂,

l) R¹¹OC(O)NR¹⁰—, and

m) C₁-C₆ alkyl, said alkyl is optionally substituted with C₁-C₆ perfluoroalkyl, R¹⁰O—, R¹⁰C(O)NR¹⁰—, R¹⁰C(O)—, R¹⁰OC(O)—, —N(R¹⁰)₂, or R¹¹OC(O)NR¹⁰—;

R^{8a} is independently selected from the group consisting of:

a) aryl,

b) C₂-C₆ alkenyl,

c) C₂-C₆ alkynyl,

d) C₁-C₆ perfluoroalkyl,

e) halo,

f) R¹⁰O—,

g) R¹⁰C(O)NR¹⁰—,

h) CN,

i) R¹⁰C(O)—,

j) R¹⁰OC(O)—,

k) —N(R¹⁰)_{2,}

l) R¹¹OC(O)NR¹⁰—, and

m) C₁-C₆ alkyl, said alkyl is optionally substituted with C₁-C₆ perfluoroalkyl, R¹⁰O—, R¹⁰C(O)NR¹⁰—, R¹⁰C(O)—, R¹⁰OC(O)—, —N(R¹⁰)₂, or R¹¹OC(O)NR¹⁰—;

R⁹ is selected from the group consisting of:

a) hydrogen,

b) halo,

c) R¹⁰O— and

d) C₁-C₆ alkyl;

R¹⁰ is hydrogen, C₁-C₈ alkyl, C₁-C₆ perfluoroalkyl, benzyl or aryl, said alkyl optionally substituted with OH or —OC₁-C₆ alkyl;

R¹¹ is C₁-C₆ alkyl or aryl;

A¹ is a bond, —CH═CH—, —C≡C—, —C(O)—, —C(O)NR¹⁰—, O, —N(R¹⁰)—, or —S(O)ₘ—;

A³ is —C(O)—, —C(R^{1a})₂—, O, —N(R¹⁰)— or S(O)ₘ;

V is:

a) heteroaryl, selected from the group consisting of imidazolyl, pyridinyl, thiazolyl, indolyl, quinolinyl, isoquinolinyl, and thienyl, or

b) aryl;

Y is aryl;

Z is aryl, said aryl optionally substituted with one or more of the following:

1) C₁₋₈ alkyl, unsubstituted or substituted with:

a) C₁₋₄ alkoxy,

b) NR⁶R⁷,

c) C₃₋₆ cycloalkyl,

d) aryl,

e) heterocyclyl,

f) OH,

g) —S(O)ₘR^{6a}, or

h) —C(O)NR⁶R⁷,

2) aryl,

3) heterocyclyl,

4) halo,

5) OR⁶,

6) NR⁶R⁷,

7) CN,

8) CF₃,

9) —S(O)ₘR^{6a},

10) —C(O)NR⁶R⁷, or

11) C₃-C₆ cycloalkyl;

m is 0, 1 or 2;

n is 0,1,2,3or 4;

p is 0, 1,2,3or 4;

r is 0,1,2,3,4,or 5;

s is 0 or 1; and

t is 0 to 5;

or a pharmaceutically acceptable salt, stereoisomer, or mixture thereof.

Yet another embodiment is exemplified by the compounds of formula C:

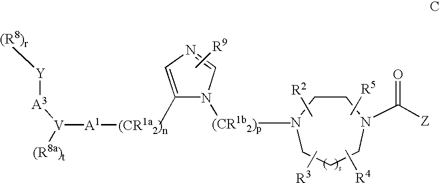

wherein:

R^{1a} and R^{1b} are independently hydrogen or C₁-C₆ alkyl, said alkyl optionally substituted with aryl, C₃-C₁₀ cycloalkyl, halo, R¹⁰O— or —N(R¹⁰)₂;

R², R³, R⁴ and R⁵ are independently selected from H and C₁₋₄ alkyl;

R⁶ and R⁷ are independently selected from the group consisting of:

a) H,

b) C₁₋₈ alkyl,

c) C₃₋₆ cycloalkyl,

d) aryl, and

e) heterocyclyl,

said alkyl, cycloalkyl, aryl, and heterocyclyl optionally substituted with:

1) C₁₋₄ alkoxy,

2) halo,

3) aryl,

4) heterocyclyl, or

5) C₁₋₄ alkyl;

R^{6a} is selected from:

a) C₁₋₄ alkyl,

b) C₃₋₆ cycloalkyl,

c) aryl, and

d) heterocyclyl,

said alkyl, cycloalkyl, aryl, and heterocyclyl optionally substituted with:

1) C₁₋₄ alkoxy,

2) halo,

3) aryl,

4) heterocyclyl, or

5) C₁₋₄ alkyl;

R⁸ is independently selected from the group consisting of:

a) aryl,

b) C₂-C₆ alkenyl,

c) C₂-C₆ alkynyl,

d) C₁-C₆ perfluoroalkyl,

e) halo,

f) R¹⁰O—,

g) R¹⁰C(O)NR¹⁰—,

h) CN,

i) R¹⁰C(O)—,

j) R¹⁰ OC(O)—,

k) —N(R¹⁰)₂,

l) R¹¹OC(O)NR¹⁰—, and

m) C₁-C₆ alkyl, said alkyl is optionally substituted with C₁-C₆ perfluoroalkyl, R¹⁰O—, R¹⁰C(O)NR¹⁰—, R¹⁰C(O)—, R¹⁰OC(O)—, —N(R¹⁰)₂, or R¹¹OC(O)NR¹⁰—;

R8a is independently selected from the group consisting of:

a) aryl,

b) C₁-C₆ alkyl,

c) C₂-C₆ alkenyl,

d) C₂-C₆ alkynyl,

e) C₁-C₆ perfluoroalkyl,

f) halo,

g) R¹⁰O—,

h) R¹⁰C(O)NR¹⁰—,

i) CN,

j) R¹⁰C(O)—,

k) R¹⁰OC(O)—,

l) —N(R¹⁰)₂,

m) R¹¹OC(O)NR¹⁰—, and

n) C₁-C₆ alkyl, said alkyl is optionally substituted with C₁-C₆ perfluoroalkyl, R¹⁰O—, R¹⁰C(O)NR¹⁰—, R¹⁰C(O)—, R¹⁰OC(O)—, —N(R¹⁰)₂, or R¹¹OC(O)NR¹⁰—;

R⁹ is selected from the group consisting of:

a) hydrogen,

b) halo,

c) R¹⁰O— and

d) C₁-C₆ alkyl;

R¹⁰ is hydrogen, C₁-C₈ alkyl, C₁-C₆ perfluoroalkyl, benzyl or aryl, said alkyl optionally substituted with OH or —OC₁-C₆ alkyl;

R¹¹ is C₁-C₆ alkyl or aryl;

A¹ is a bond, —CH═CH—, —C≡C—, —C(O)—, —C(O)NR¹⁰—, O, —N(R¹⁰)—, or —S(O)ₘ—;

A³ is —C(O)—, —C(R^{1a})₂—, O, —N(R¹⁰)— or S(O)ₘ;

V is:

a) heteroaryl, selected from the group consisting of imidazolyl, pyridinyl, thiazolyl, indolyl, quinolinyl, isoquinolinyl, and thienyl, or

b) aryl;

Y is aryl;

Z is aryl, said aryl optionally substituted with one or more of the following:

1) C₁₋₈ alkyl, unsubstituted or substituted with:

a) C₁₋₄ alkoxy,

b) NR⁶R⁷,

c) C₃₋₆ cycloalkyl,

d) aryl,

e) heterocyclyl,

f) OH,

g) —S(O)ₘR^{6a}, or

h) —C(O)NR⁶R⁷,

2) aryl,

3) heterocyclyl,

4) halo,

5) OR⁶,

6) NR⁶R⁷,

7) CN,

8) CF₃,

9) —S(O)ₘR^{6a},

10) —C(O)NR⁶R⁷, or

11) C₃-C₆ cycloalkyl;

m is 0,1 or 2;

n is 0, 1, or 2;

p is 0,1,or 2;

r is 1 to 3;

s is 1;and

t is 0to 3;

or a pharmaceutically acceptable salt, stereoisomer, or mixture thereof.

A further embodiment of the present invention is a compound of Formula D:

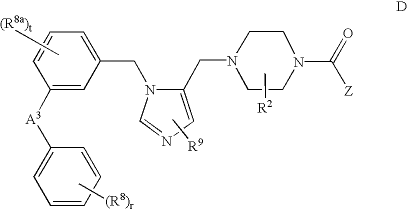

wherein

R² is H or C₁₋₄ alkyl;

R⁸ is CN, halo, C₁₋₆ alkyl, or CF₃;

R^{8a} is OR¹⁰, CN, halo, C₁₋₆ alkyl, or CF₃;

R⁹ is H or C₁₋₃ alkyl;

R¹⁰ is H, C₁₋₈ alkyl, C₁₋₆ perfluoroalkyl, benzyl, or aryl, said alkyl optionally substituted with OH or OC₁₋₈ alkyl;

A³ is O or S(O)ₘ;

Z is aryl, said aryl optionally substituted with one, two or three substituents selected from:

1) C₁₋₈ alkyl,

2) aryl,

3) heterocyclyl,

4) halo,

5) OH,

6) CN,

7) OC₁₋₆ alkyl, and

8) CF₃;

m is 0, 1, or 2; and r and t are independently 0, 1, or 2.

Specific examples of the compounds of this invention are the following:

1-(2-hydroxy-5-methylbenzoyl)-4-[1-(3-((3 -(2-hydroxyethoxy)phenyl)oxy)-4-cyanobenzyl)-2-methyl-5-imidazolylmethyl]piperazine;

1-(2-methoxy-5-methylbenzoyl)-4-[1-(3-((3-(2-hydroxyethoxy)phenyl)oxy)-4-cyanobenzyl)-2-methyl-5-imidazolylmethyl]piperazine;

1-(2-butoxy-5-methylbenzoyl)-4-[1-(3-((3-(2-hydroxyethoxy)phenyl)oxy)-4-cyanobenzyl)-2-methyl-5-imidazolylmethyl]piperazine;

1-(2-methoxybenzoyl)-4-[1-(3-((3-(2-hydroxyethoxy)phenyl)oxy)-4-cyanobenzyl)-2-methyl-5 -imidazolylmethyl]piperazine;

1 -(2-butoxybenzoyl)-4-[ 1-(3 -((3-(2-hydroxyethoxy)phenyl)oxy)-4-cyanobenzyl)-2-methyl-5-imidazolylmethyl]piperazine;

1-(2-methoxy-4-methylbenzoyl)-4-[1-(3-((3-(2-hydroxyethoxy)phenyl)oxy)-4-cyanobenzyl)-2-methyl-5-imidazolylmethyl]piperazine;

1-(2-butoxy-4-methylbenzoyl)-4-[1-(3-((3-(2-hydroxyethoxy)phenyl)oxy)-4-cyanobenzyl)-2-methyl-5 -imidazolylmethyl]piperazine;

1-(2-methoxy-3-methylbenzoyl)-4-[1-(3-((3-(2-hydroxyethoxy)phenyl)oxy)-4-cyanobenzyl)-2-methyl-5-imidazolylmethyl]piperazine;

1 -(2-butoxy-3 -methylbenzoyl)-4-[1-(3 -((3-(2-hydroxyethoxy)phenyl)oxy)-4cyanobenzyl)-2-methyl-5-imidazolylmethyl]piperazine;

or the pharmaceutically acceptable salts or optical isomers thereof.

Particular examples of compounds of this invention are: 1 -(2-butoxybenzoyl)-4-[1 -(3-((3 -(2-hydroxyethoxy)phenyl)oxy)-4-cyanobenzyl)-2-methyl-5-imidazolylmethyl]piperazine, 1-(2-methoxy-4-methylbenzoyl)-4-[1-(3-((3-(2-hydroxyethoxy)phenyl)oxy)-4-cyanobenzyl)-2-methyl-5-imidazolylmethyl] piperazine, and pharmaceutically acceptable salts thereof.

Also within the scope of the invention is 1-(tert-butoxycarbonyl)-4-[1 -(3-((3-(2-hydroxyethoxy)phenyl)oxy)-4-cyanobenzyl)-2-methyl-5-imidazolyl-methyl]piperazine.

The compounds of the instant invention differ from previously disclosed piperazine-containing compounds, (PCT Publication No. WO 96/30343—Oct. 3, 1996; U.S. Pat. No. 5,856,326; PCT Publication No. WO 96/31501—Oct. 10, 1996; PCT Publication No. WO 97/36593 —Oct. 9, 1997; PCT Publication No. WO 97/36592—Oct. 9, 1997) that were described as inhibitors of farnesyl-protein transferase (FPTase), in that, among other things, the instant compounds are dual inhibitors of farnesyl-protein transferase and geranylgeranyl-protein transferase type I (GGTase-I).

The compounds of the instant invention are further characterized in that the inhibitory activity of the compounds against FPTase is greater than the inhibitory activity against GGTase-I. Preferably, the compounds of the instant invention inhibit FPTase in vitro (Example 10) at an IC₅₀ of less than 100 nM and inhibit GGTase-I in vitro (Example 11) at an IC₅₀ of less than 5 μM. Preferably, the compounds of the instant invention inhibit the cellular processing of the hDJ protein (Example 15) at an EC₅₀ of less than about 250 nM. Also preferably, the compounds of the instant invention inhibit the cellular processing of the Rap1 protein (Example 16) at an EC₅₀ of less than about 10 μM. More preferably, the compounds of the instant invention inhibit the cellular processing of the Rap1 protein (Example 16) at an EC₅₀ of less than about 1 μM. Also more preferably, the ratio of the IC₅₀ of the compounds of this embodiment of the instant invention for in vitro inhibition of GGTase type I to the IC₅₀ of the compounds of the instant invention for in vitro inhibition of FPTase is greater than 1 and less than 25. Also more preferably, the ratio of the EC₅₀ of the compounds of the instant invention for inhibition of the cellular processing of the hDJ protein (Example 15) to the EC₅₀ of the compounds of the instant invention for inhibition of the cellular processing of the Rap1 protein is between about 1 and about 100.

The compounds of the present invention may have asymmetric centers and occur as racemates, racemic mixtures, and as individual diastereomers, with all possible isomers, including optical isomers, being included in the present invention. When any variable (e.g. aryl, heterocycle, R¹, R² etc.) occurs more than one time in any constituent, its definition on each occurrence is independent at every other occurrence. Also, combinations of substituents/or variables are permissible only if such combinations result in stable compounds.

As used herein, “alkyl” is intended to include both branched and straight-chain saturated aliphatic hydrocarbon groups having the specified number of carbon atoms; “alkoxy” represents an alkyl group of indicated number of carbon atoms attached through an oxygen bridge. “Alkenyl” is intended to include both branched and straight-chain unsaturated aliphatic hydrocarbon groups having the specified number of carbon atoms. “Halogen” or “halo” as used herein means fluoro, chloro, bromo and iodo.

As used herein, “cycloalkyl” is intended to include monocyclic saturated aliphatic hydrocarbon groups having the specified number of carbon atoms. Examples of such cycloalkyl groups includes, but are not limited to, cyclopropyl, cyclobutyl, cyclohexyl, cycloheptyl and cyclooctyl.

As used herein, “aryl” is intended to mean any stable monocyclic or bicyclic carbon ring of up to 7 members in each ring, wherein at least one ring is aromatic. Examples of such aryl elements include phenyl, naphthyl, tetrahydronaphthyl, indanyl, biphenyl, phenanthryl, anthryl or acenaphthyl.

The term heterocycle or heterocyclic, as used herein, represents a stable 5- to 7-membered monocyclic or stable 8- to 11-membered bicyclic hetero-cyclic ring which is either saturated or unsaturated, and which consists of carbon atoms and from one to four heteroatoms selected from the group consisting of N, O, and S, and including any bicyclic group in which any of the above-defined heterocyclic rings are fused to a benzene ring. The term heterocycle or heterocyclic includes heteroaryl moieties. The heterocyclic ring may be attached at any hetero-atom or carbon atom which results in the creation of a stable structure. Examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzo-furyl, dihydrobenzothienyl, dihydrobenzothiopyranyl, dihydrobenzothiopyranyl sulfone, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperdinyl, 2-oxopyrrolidinyl, piperidyl, piperazinyl, pyridyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, and thienyl.

As used herein, “heteroaryl” is intended to mean any stable monocyclic or bicyclic carbon ring of up to 7 members in each ring, wherein at least one ring is aromatic and wherein from one to four carbon atoms are replaced by heteroatoms selected from the group consisting of N, O, and S. Examples of such heterocyclic elements include, but are not limited to, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, furyl, imidazolyl, indolinyl, indolyl, isochromanyl, isoindolinyl, isoquinolinyl, isothiazolyl, naphthyridinyl, oxadiazolyl, pyridyl, pyridyl N-oxide, pyrazinyl, pyrazolyl, pyridazinyl, pyrimidinyl, pyrrolyl, quinazolinyl, quinolinyl, quinoxalinyl, tetrahydroisoquinolinyl, tetrahydroquinolinyl, thiazolyl, thienofuryl, thienothienyl, and thienyl.

As used herein in the definition of R² and R³, the term “the substituted group” is intended to mean a substituted C₁₋₈ alkyl, substituted C₂₋₈ alkenyl, substituted C₂₋₈ alkynyl, substituted aryl or substituted heterocycle from which the substituent(s) R² and R³ are selected.

As used herein in the definition of R⁶, R^{6a}, R⁷ and R^{7a}, the substituted C₁₋₄ alkyl, substituted C₃₋₆ cycloalkyl, substituted aroyl, substituted aryl, substituted heteroaroyl, substituted arylsulfonyl, substituted heteroarylsulfonyl and substituted heterocycle include moieties containing from 1 to 3 substituents in addition to the point of attachment to the rest of the compound.

When R⁶ and R⁷ or R⁷ and R^{7a} are joined to form a ring, examples of such rings include, but are not limited to:

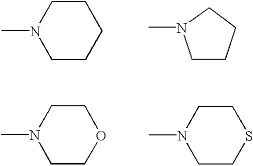

When R² and R³ are combined to form —(CH₂)ᵤ—, cyclic moieties are formed. Examples of such cyclic moieties include, but are not limited to:

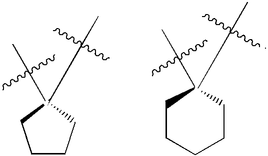

In addition, such cyclic moieties may optionally include a heteroatom(s). Examples of such heteroatom-containing cyclic moieties include, but are not limited to:

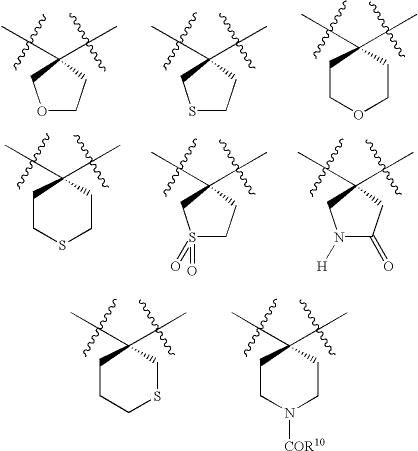

Lines drawn into the ring systems from substituents (such as from R², R³, R⁴ etc.) indicate that the indicated bond may be attached to any of the substitutable ring carbon atoms. When more than one substituent is present on a ring system, the substituents may be bonded to the same carbon as long as a stable structure results.

Preferably, R^{1a} and R^{1b} are independently selected from: hydrogen, —N(R¹⁰)₂, (R¹⁰)₂NC(O)—, R¹⁰C(O)NR¹⁰— or unsubstituted or substituted C₁-C₆ alkyl wherein the substituent on the substituted C₁-C₆ alkyl is selected from unsubstituted or substituted phenyl, —N(R¹⁰)₂, R¹⁰O— and R¹⁰C(O)NR¹⁰—.

Preferably, R² is selected from: hydrogen,

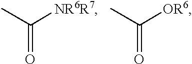

and an unsubstituted or substituted group, the group selected from C₁₋₈ alkyl, C₂₋₈ alkenyl and C₂₋₈ alkynyl;

wherein the substituted group is substituted with one or more of:

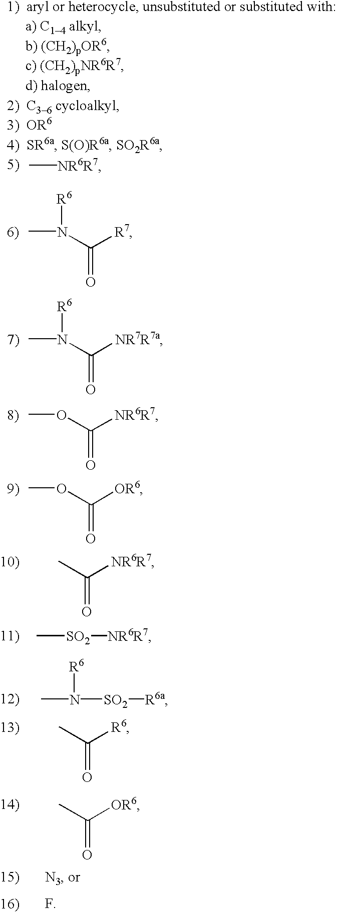

Preferably R³ is selected from H and C₁-C₆ alkyl.

Preferably, R⁴ is hydrogen.

Preferably, R⁵ is hydrogen.

Preferably, R⁶, R⁷ and R^{7a} are selected from: hydrogen, unsubstituted or substituted C₁-C₄ alkyl, unsubstituted or substituted aryl and unsubstituted or substituted cycloalkyl.

Preferably, R^{6a} is unsubstituted or substituted C₁-C₆ alkyl, unsubstituted or substituted aryl and unsubstituted or substituted cycloalkyl.

Preferably, R⁹ is hydrogen, chloro, R¹⁰O— or C₁-C₆ alkyl.

Preferably, R¹⁰ is selected from H, C₁-C₆ alkyl, hydroxyalkyl, alkoxyalkyl, benzyl and aryl.

Preferably, A¹ and A² are independently selected from: a bond, —C(O)NR¹⁰—, —NR¹⁰C(O)—, O, —N(R¹⁰)—, —S(O)₂N(R¹⁰)— and —N(R¹⁰)S(O)₂—. Most preferably, A¹ and A² are a bond.

Preferably, A³ is selected from: —O— and S(O)ₘ.

Preferably, V is selected from heteroaryl and aryl.

More preferably, V is phenyl or pyridyl.

Preferably, W is selected from imidazolyl, oxazolyl, pyrazolyl, pyyrolidinyl, pyridinyl, thiazolyl, indolyl, quinolinyl, and isoquinolinyl. More preferably W is selected from imidazolyl and pyridinyl.

Preferably, Y is phenyl.

Preferably, Z is selected from unsubstituted or substituted phenyl, unsubstituted or substituted napthyl, unsubstituted or substituted pyridyl and unsubstituted or substituted quinoline.

More preferably, Z is unsubstituted or substituted phenyl or unsubstituted or substituted pyridyl wherein the substituted phenyl or substituted pyridyl are substituted with one or more of the following:

a) OH,

b) alkoxy,

c) aryloxy,

d) C₁-C₄ alkyl,

e) NO₂,

f) halogen,

g) CF₃,

h) SO₂CH₃, or

i) R¹⁰O—;

Preferably, n and r are independently 0, 1, or 2.

Preferably p is 1, 2 or 3.

Preferably s is 0.

Preferably, the moiety

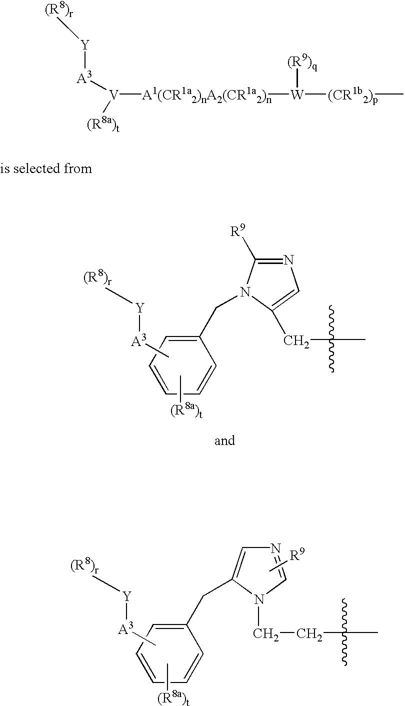

Preferably, the moiety —A¹(CR^{1a}₂)ₙA²(CR^{1a}₂)ₙ— is not a bond.

It is intended that the definition of any substituent or variable (e.g., R^{1a}, R⁹, n, etc.) at a particular location in a molecule be independent of its definitions elsewhere in that molecule. Thus, —N(R¹⁰)₂ represents —NHH, —NHCH₃, —NHC₂H₅, etc. It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials.

The pharmaceutically acceptable salts of the compounds of this invention include the conventional non-toxic salts of the compounds of this invention as formed, e.g., from non-toxic inorganic or organic acids. For example, such conventional non-toxic salts include those derived from inorganic acids such as hydrochloric, hydrobromic, sulfuric, sulfamic, phosphoric, nitric and the like: and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like.

The pharmaceutically acceptable salts of the compounds of this invention can be synthesized from the compounds of this invention which contain a basic moiety by conventional chemical methods. Generally, the salts are prepared either by ion exchange chromatography or by reacting the free base with stoichiometric amounts or with an excess of the desired salt-forming inorganic or organic acid in a suitable solvent or various combinations of solvents.

Reactions used to generate the compounds of this invention are prepared by employing reactions as shown in the Schemes 1-16, in addition to other standard manipulations such as ester hydrolysis, cleavage of protecting groups, etc., as may be known in the literature or exemplified in the experimental procedures. Substituents R, R^{a} and R^{b}, as shown in the Schemes, represent the substituents R², R³ and R⁴; however their point of attachment to the ring is illustrative only and is not meant to be limiting.

These reactions may be employed in a linear sequence to provide the compounds of the invention or they may be used to synthesize fragments which are subsequently joined by the alkylation reactions described in the Schemes.

Synopsis of Schemes 1-16:

The requisite intermediates are in some cases commercially available, or can be prepared according to literature procedures, for the most part. In Scheme 1, for example, the synthesis of 2-alkyl substituted piperazines is outlined, and is essentially that described by J. S. Kiely and S. R. Priebe in Organic Preparations and Proceedings Int., 1990, 22, 761-768. Boc-protected amino acids (1), available commercially or by procedures known to those skilled in the art, can be coupled to N-benzyl amino acid esters using a variety of dehydrating agents such as DCC (dicyclohexycarbodiimide) or EDC HCl (1-ethyl-3-(3-dimethylaminopropyl) carbodiimide hydrochloride) in a solvent such as methylene chloride, chloroform, dichloroethane, or in dimethylformamide. The product (II) is then deprotected with acid, for example hydrogen chloride in chloroform or ethyl acetate, or trifluoroacetic acid in methylene chloride, and cyclized under weakly basic conditions to give the diketopiperazine (III). Reduction of (III) with lithium aluminum hydride in refluxing ether gives the piperazine (IV), which is protected as the Boc derivative (V). The N-benzyl group can be cleaved under standard conditions of hydrogenation, e.g., 10% palladium on carbon at 60 psi hydrogen on a Parr apparatus for 24-48 h. The product (VI) can be coupled (Scheme 2) to a carboxylic acid under standard conditions to furnish amides (VII); a final acid deprotection as previously described gives the intermediate (VIII) (Scheme 2).

Scheme 3 sets forth the preparation of fluorobenzonitrilealdehyde (XIII). 4-bromo-3-fluorotoluene (IX) in DMF is reacted with Zn(CN)₂ and Pd(PPh₃)₄. The resulting product is treated with N-bromosuccinamide and benzoylperoxide to give 4-cyano-3-fluorobenzylbromide (X). Acetoxymethyl-imidazole (XI) is prepared by combining (X) with a protected imidazole acetate in EtOAc at reflux. Although the scheme exemplifies the reaction with a cyano substituted aryl, other electron withdrawing groups can be utilized as will be readily apparent to those skilled in the art. The acetate (XI) is hydrolized to the corresponding alcohol with LiOH/water and oxidized to aldehyde (XIII) under standard oxidation conditions. Aldehyde (XIII) can be reductively alkylated with a variety of amines such as piperazine (VIII) (Scheme 4). The resulting intermediates such as (XIV) can be converted into final products (XV) via base-promoted addition reactions as depicted in Scheme 4.

Scheme 5 depicts a method for synthesizing substituted imidazole aldehydes (XVII) in which 4-cyano-3-fluorobenzylbromide (X) in DMF is reacted with commercially available 4-formyl-2-methylimidazole (XVI) and Cs₂CO₃. These substituted imidazole aldehydes (XVII) can be converted to compounds of the instant invention (XXII) as depicted in Scheme 6.

As shown in Scheme 7, the piperazine intermediate (VIII) can be reductively alkylated with other aldehydes such as 1-trityl-4-imidazolylarboxaldehyde or 1-trityl-4-imidazolylacetaldehyde, to give products such as (XXIII). The trityl protecting group can be removed from (XXIII) to give (XXIV), or alternatively, (XXIII) can first be treated with an alkyl halide then subsequently deprotected to give the alkylated imidazole (XXV).

Access to alternatively substituted piperazines is described in Scheme 8. Following deprotection with trifluoroacetic acid, N-benzyl piperazine (V) can be coupled to with a carboxylic acid under standard conditions to give N-benzyl amide (XXVII). The resulting N-benzyl amide (XXVII) can be hydrogenated in the presence of a catalyst to give the piperazine (XXVII) which can then be carried on to final products as described, for example, in Schemes 4 and 6.

Scheme 9 provides an illustrative example of the synthesis of compounds of the instant invention wherein the substituents R² and R³ are combined to form —(CH₂)ᵤ—. For example, 1-aminocyclohexane-1-carboxylic acid (XXIX) can be converted to the spiropiperazine (XXXV) according to the procedures outlined in Scheme 9. The piperazine intermediate can be coupled to a carboxylic acid to give (XXXVI), reductively alkylated to give (XXXVII) and deprotected under standard conditions to give (XXXVIII). It is understood that the imidazolylalkyl substituent may be readily replaced by other reagents well known in the art and readily available to provide other N-substituents on the piperazine.

Scheme 10 depicts another procedure for obtaining compounds of the instant invention. Reductive alkylation of substituted piperazine (VIII) with a protected imidazole carboxaldehyde leads to (XXXIX), which can be alkylated with an arylmethylhalide to give the imidazolium salt (XL). Final removal of protecting groups by either solvolysis with a lower alkyl alcohol, such as methanol, or treatment with triethylsilane in methylene chloride in the presence of trifluoroacetic acid gives the final product (XLI).

Amino acids of the general formula (XLIII) which have a sidechain not found in natural amino acids may be prepared by the reactions illustrated in Scheme 11 starting with the readily prepared imine (XLII).

Schemes 12-15 illustrate syntheses of suitably substituted aldehydes useful in the syntheses of the instant compounds wherein the variable W is present as a pyridyl moiety. Similar synthetic strategies for preparing alkanols that incorporate other heterocyclic moieties for variable W are also well known in the art.

Scheme 16 illustrates the synthetic strategy that is employed when the R⁸ substitutent is not an electronic withdrawing moiety either ortho or para to the fluorine atom. In the absence of the electronic withdrawing moiety, the alkylation can be accomplished via an Ullmann reaction. Thus, the imidazolylmethylacetate (XLIV) is treated with a suitably substituted halobenzylbromide to provide the 1-benzylimidazolyl intermediate (XLV). The acetate functionality of intermediate (XLV) is converted to an aldehyde which is then reductively coupled to intermediate (VIII), prepared as illustrated in Scheme 2. Coupling under standard Ullmann conditions provided compound (XLVII) of the instant invention.

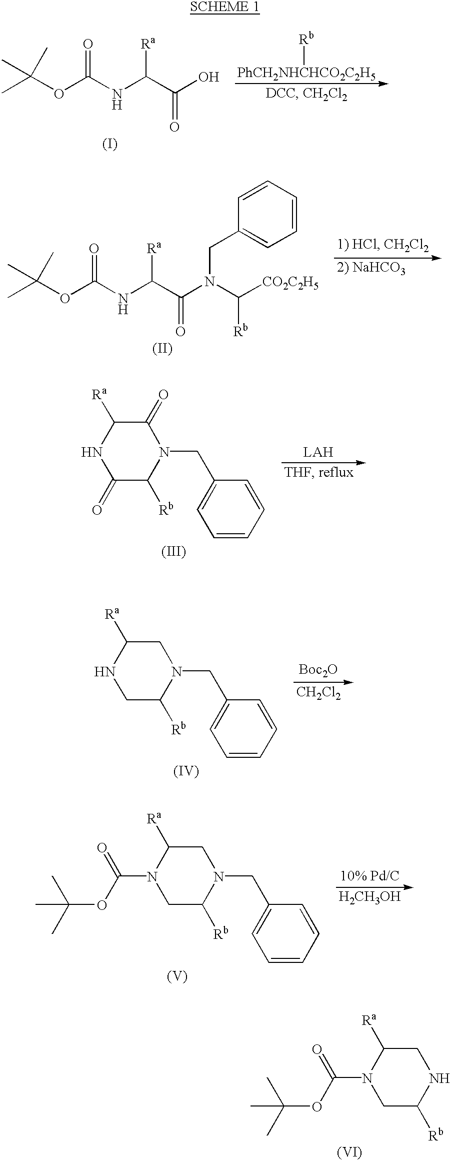

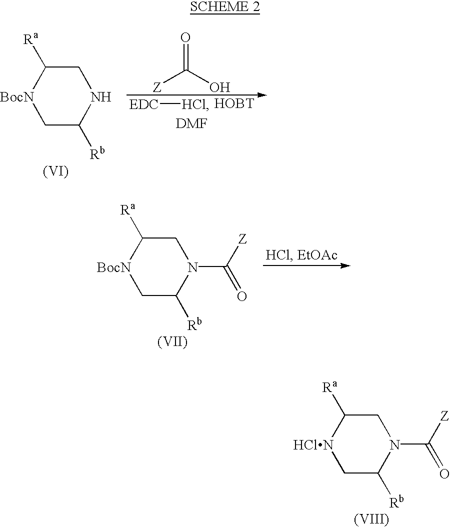

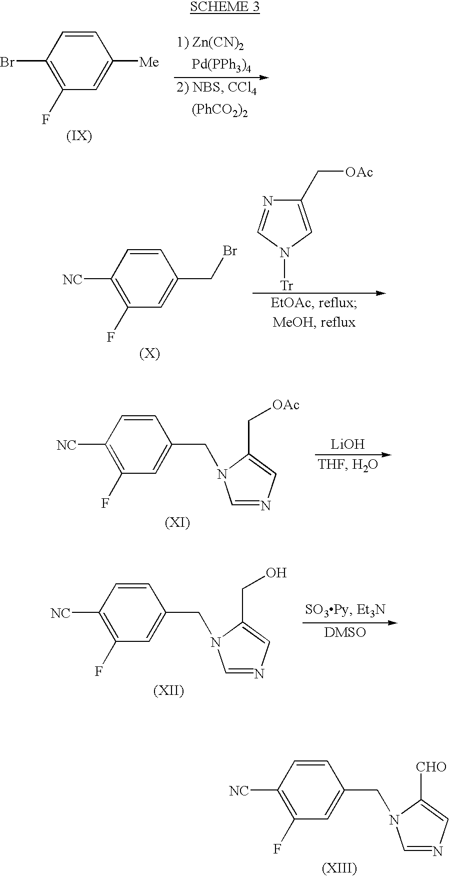

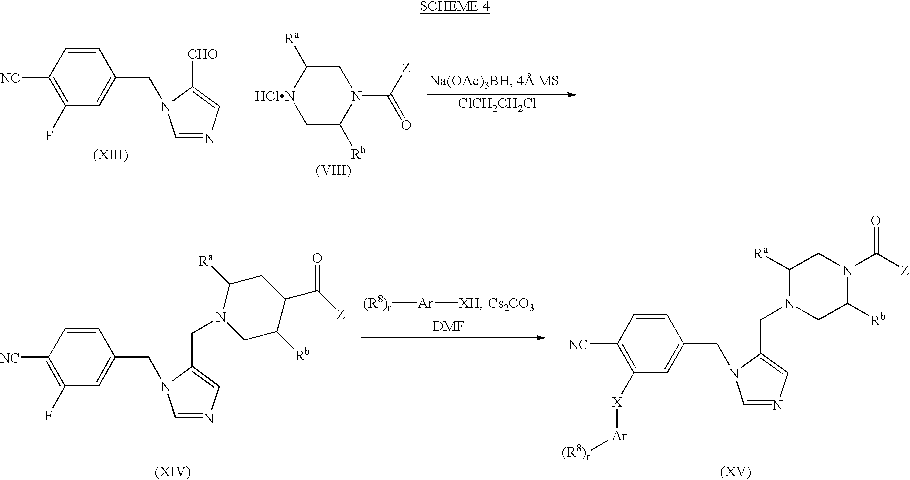

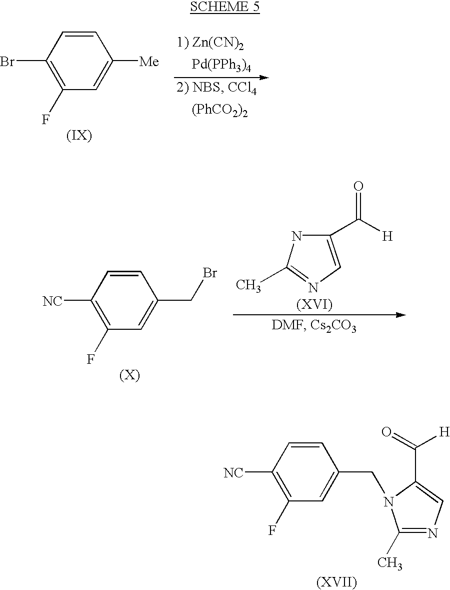

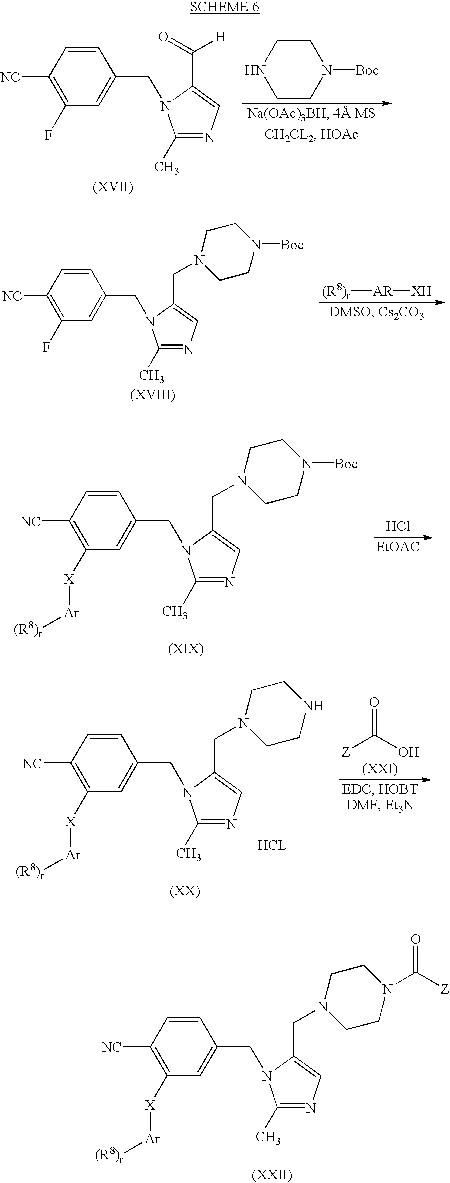

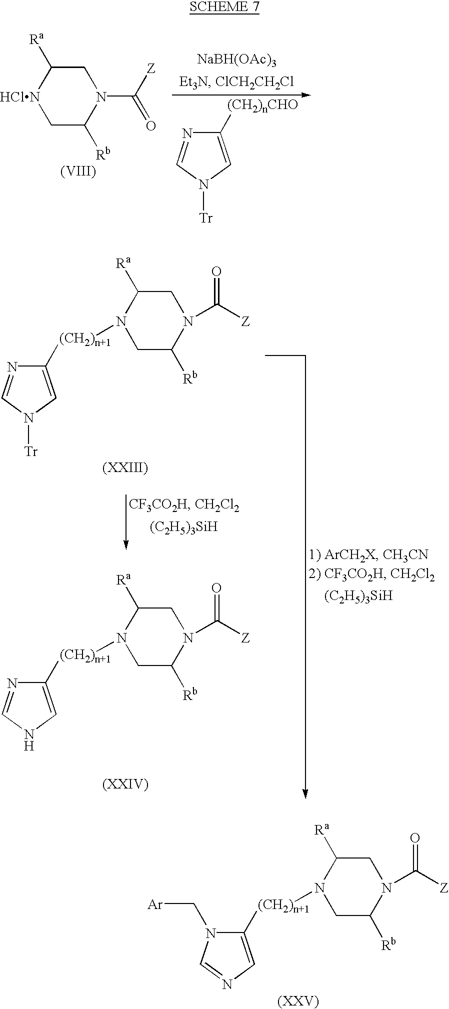

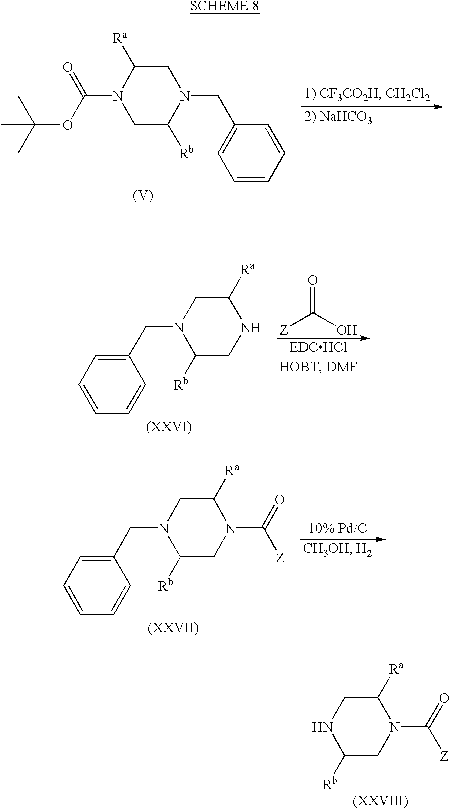

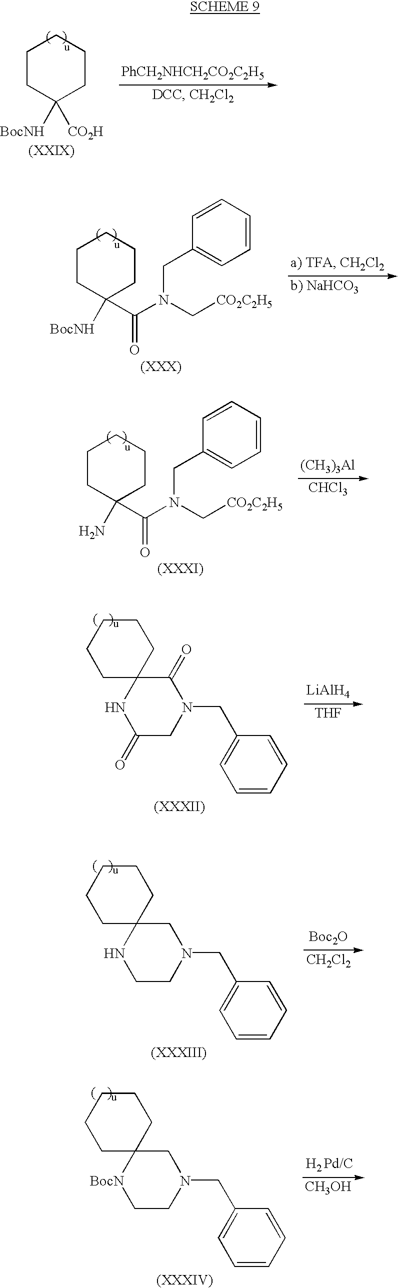

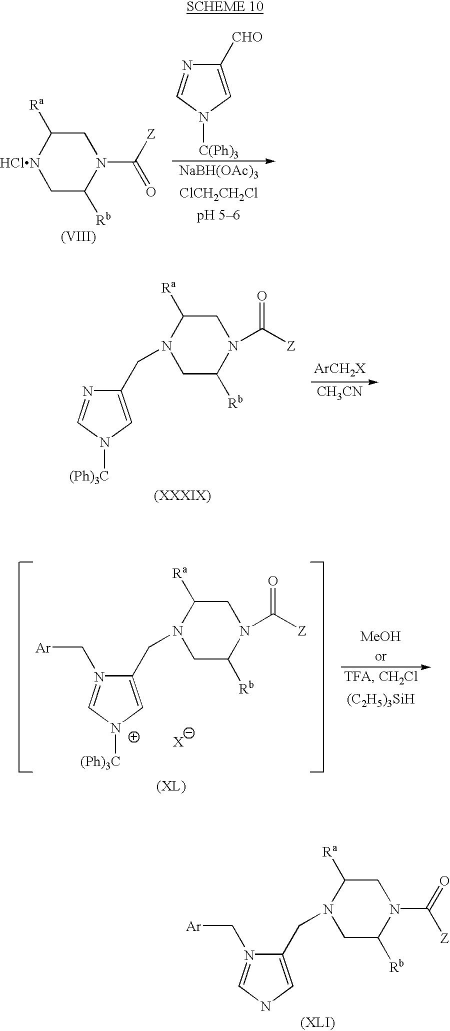

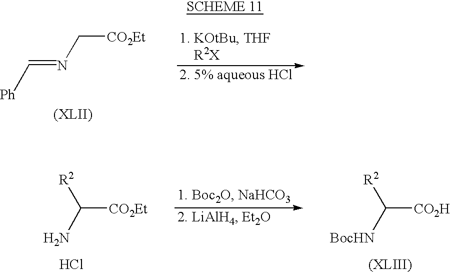

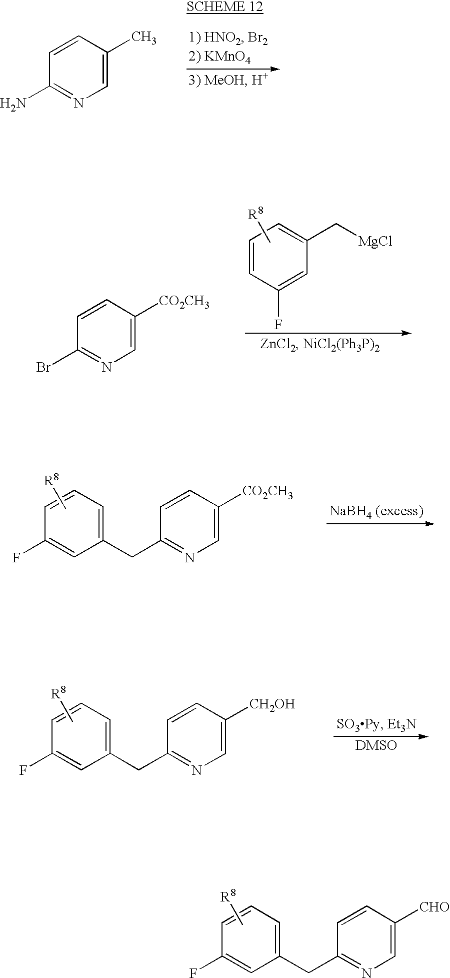

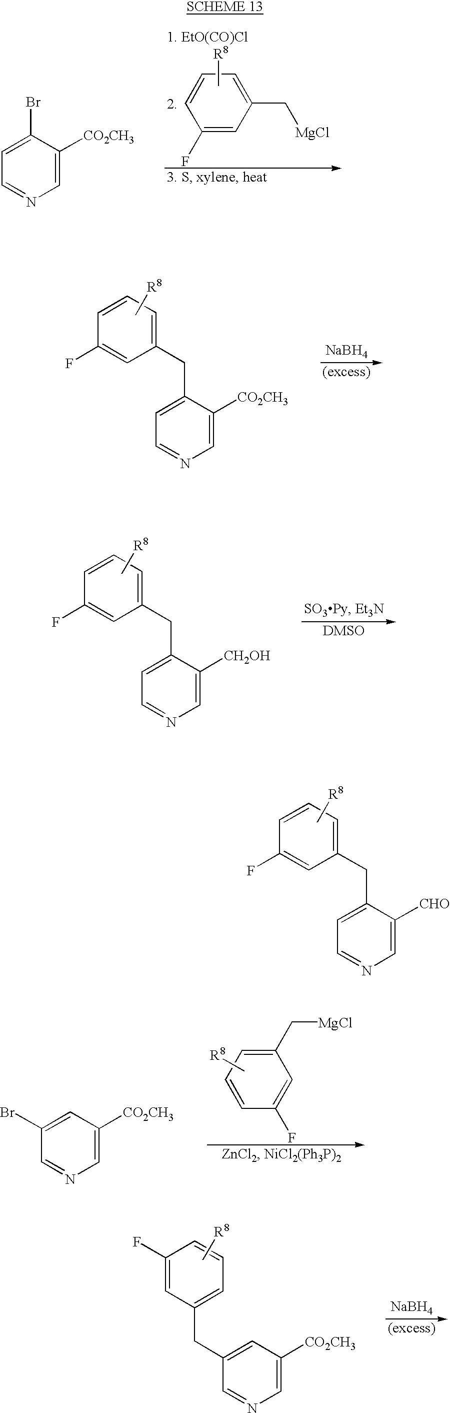

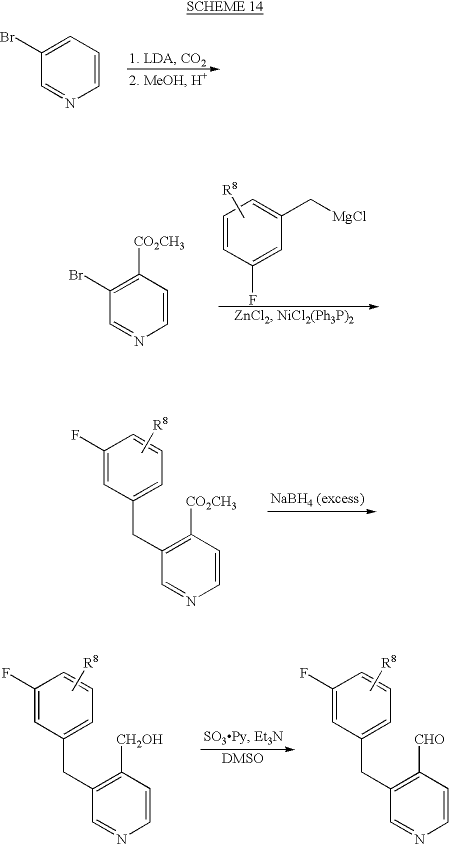

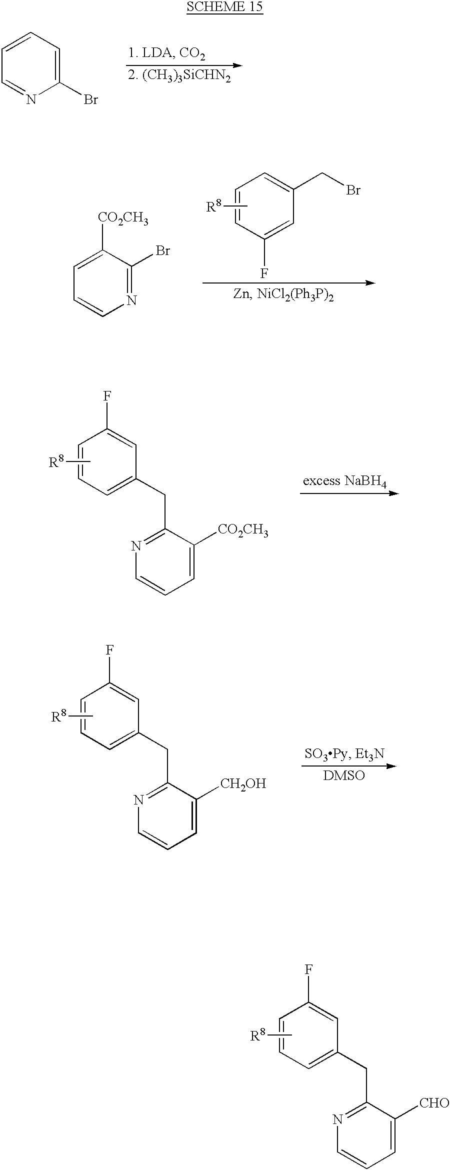

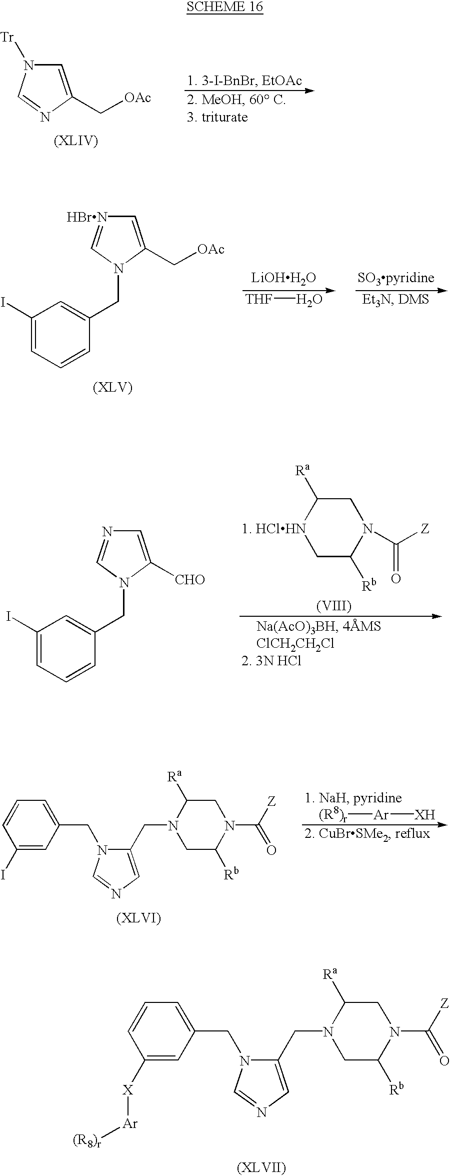

The instant compounds are useful as pharmaceutical agents for mammals, especially for humans. These compounds may be administered to patients for use in the treatment of cancer. Examples of the type of cancer which may be treated with the compounds of this invention include, but are not limited to, colorectal carcinoma, exocrine pancreatic carcinoma, myeloid leukemias and neurological tumors. Such tumors may arise by mutations in the ras genes themselves, mutations in the proteins that can regulate Ras activity (i.e., neurofibromin (NF-1), neu, src, abl, lck, fyn) or by other mechanisms.

The compounds of the instant invention inhibit prenyl-protein transferase and the prenylation of the oncogene protein Ras. The instant compounds may also inhibit tumor angiogenesis, thereby affecting the growth of tumors (J. Rak et al. Cancer Research, 55: 4575-4580 (1995)). Such anti-angiogenesis properties of the instant compounds may also be useful in the treatment of certain forms of vision deficit related to retinal vascularization.

The compounds of this invention are also useful for inhibiting other proliferative diseases, both benign and malignant, wherein Ras proteins are aberrantly activated as a result of oncogenic mutation in other genes (i.e., the Ras gene itself is not activated by mutation to an oncogenic form) with said inhibition being accomplished by the administration of an effective amount of the compounds of the invention to a mammal in need of such treatment. For example, a component of NF-1 is a benign proliferative disorder.

The instant compounds may also be useful in the treatment of certain viral infections, in particular in the treatment of hepatitis delta and related viruses (J. S. Glenn et al. Science, 256:1331-1333 (1992).

The compounds of the instant invention are also useful in the prevention of restenosis after percutaneous transluminal coronary angioplasty by inhibiting neointimal formation (C. Indolfi et al. Nature medicine, 1:541-545(1995).

The instant compounds may also be useful in the treatment and prevention of polycystic kidney disease (D. L. Schaffner et al. American Journal of Pathology, 142:1051-1060 (1993) and B. Cowley, Jr. et al.FASEB Journal, 2:A3160 (1988)).

The instant compounds may also be useful for the treatment of fungal infections.

The instant compounds may also be useful as inhibitors of proliferation of vascular smooth muscle cells and therefore useful in the prevention and therapy of arteriosclerosis and diabetic vascular pathologies.

The compounds of this invention may be administered to mammals, preferably humans, either alone or, preferably, in combination with pharmaceutically acceptable carriers, excipients or diluents, in a pharmaceutical composition, according to standard pharmaceutical practice. The compounds can be administered orally or parenterally, including the intravenous, intramuscular, intraperitoneal, subcutaneous, rectal and topical routes of administration.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropylmethylcellulose or hydroxypropyl-cellulose, or a time delay material such as ethyl cellulose, cellulose acetate buryrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of Formula A may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound of Formula A are employed. (For purposes of this application, topical application shall include mouth washes and gargles.) The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

As used herein, the term “composition” is intended to encompass a product comprising the specified ingredients in the specific amounts, as well as any product which results, directly or indirectly, from combination of the specific ingredients in the specified amounts.

When a compound according to this invention is administered into a human subject, the daily dosage will normally be determined by the prescribing physician with the dosage generally varying according to the age, weight, sex and response of the individual patient, as well as the severity of the patient's symptoms.

In one exemplary application, a suitable amount of compound is administered to a mammal undergoing treatment for cancer. Administration occurs in an amount between about 0.1 mg/kg of body weight to about 60 mg/kg of body weight per day, preferably of between 0.5 mg/kg of body weight to about 40 mg/kg of body weight per day.

The compounds of the instant invention may also be co-administered with other well known therapeutic agents that are selected for their particular usefulness against the condition that is being treated. For example, the compounds of the instant invention may also be co-administered with other well known cancer therapeutic agents that are selected for their particular usefulness against the condition that is being treated. Included in such combinations of therapeutic agents are combinations of the instant prenyl-protein transferase inhibitors and an antineoplastic agent. It is also understood that such a combination of antineoplastic agent and inhibitor of prenyl-protein transferase may be used in conjunction with other methods of treating cancer and/or tumors, including radiation therapy and surgery.

Examples of an antineoplastic agent include, in general, microtubule-stabilizing agents (such as paclitaxel (also known as Taxol®), docetaxel (also known as Taxotere®), epothilone A, epothilone B, desoxyepothilone A, desoxyepothilone B or their derivatives); microtubule-disruptor agents; alkylating agents, anti-metabolites; epidophyllotoxin; an antineoplastic enzyme; a topoisomerase inhibitor; procarbazine; mitoxantrone; platinum coordination complexes; biological response modifiers and growth inhibitors; hormonal/anti-hormonal therapeutic agents, haematopoietic growth factors and antibodies (such as trastuzumab (Herceptin™)).

Example classes of antineoplastic agents include, for example, the anthracycline family of drugs, the vinca drugs, the mitomycins, the bleomycins, the cytotoxic nucleosides, the taxanes, the epothilones, discodermolide, the pteridine family of drugs, diynenes and the podophyllotoxins. Particularly useful members of those classes include, for example, doxorubicin, carminomycin, daunorubicin, aminopterin, methotrexate, methopterin, dichloro-methotrexate, mitomycin C, porfiromycin, 5-fluorouracil, 6-mercaptopurine, gemcitabine, cytosine arabinoside, podophyllotoxin or podo-phyllotoxin derivatives such as etoposide, etoposide phosphate or teniposide, melphalan, vinblastine, vincristine, leurosidine, vindesine, leurosine, paclitaxel and the like. Other useful antineoplastic agents include estramustine, cisplatin, carboplatin, cyclophosphamide, bleomycin, tamoxifen, ifosamide, melphalan, hexamethyl melamine, thiotepa, cytarabin, idatrexate, trimetrexate, dacarbazine, L-asparaginase, camptothecin, CPT-11, topotecan, ara-C, bicalutamide, flutamide, leuprolide, pyridobenzoindole derivatives, interferons and interleukins.

The preferred class of antineoplastic agents is the taxanes and the preferred antineoplastic agent is paclitaxel.

The instant compounds may also be useful in combination with prodrugs of antineoplastic agents. In particular, the instant compounds may be co-administered, either concurrently or sequentially, with a conjugate (termed a “PSA conjugate”) which comprises an oligopeptide, that is selectively cleaved by enzymatically active prostate specific antigen (PSA), and an antineoplastic agent. Such co-administration will be particularly useful in the treatment of prostate cancer or other cancers which are characterized by the presence of enzymatically active PSA in the immediate surrounding of the cancer cells, that PSA which is secreted by the cancer cells.

Compounds which are PSA conjugates and are therefore useful in such a co-administration, and methods of synthesis thereof, can be found in the following patents, pending applications and publications, which are herein incorporated by reference: U.S. Pat. No. 5,599,686 granted on Feb. 4, 1997; WO 96/00503 (Jan. 11, 1996); U.S. Ser. No. 08/404,833 filed on Mar. 15, 1995; U.S. Ser. No. 08/468,161 filed on Jun. 6, 1995; U.S. Pat. No. 5,866,679 granted on Feb. 2, 1999; WO 98/10651 (Mar. 19, 1998); U.S. Ser. No. 08/926,412 filed on Sep. 9, 1997; WO 98/18493 (May 7, 1998); U.S. Ser. No. 08/950,805 filed on Oct. 14, 1997; U.S. Ser. No. 09/112,656 filed on Jul. 9, 1998; U.S. Ser. No. 60/052,195 filed on Jul. 10, 1997; and U.S. Ser. No. 09/193,365 filed on Nov. 17, 1998; U.S. Ser. No. 60/067,110 filed on Dec. 2, 1997.

Compounds which are described as prodrugs wherein the active therapeutic agent is release by the action of enzymatically active PSA and therefore may be useful in such a co-administration, and methods of synthesis thereof, can be found in the following patents, pending applications and publications, which are herein incorporated by reference: WO 98/52966 (Nov. 26, 1998).

All patents, publications and pending patent applications identified are hereby incorporated by reference.

Radiation therapy, including x-rays or gamma rays which are delivered from either an externally applied beam or by implantation of tiny radioactive sources, may also be used in combination with the instant inhibitor of prenyl-protein transferase alone to treat cancer.

Additionally, compounds of the instant invention may also be useful as radiation sensitizers, as described in WO 97/38697, published on Oct. 23, 1997, and herein incorporated by reference.

The instant compounds may also be useful in combination with other inhibitors of parts of the signaling pathway that links cell surface growth factor receptors to nuclear signals initiating cellular proliferation. Thus, the instant compounds may be utilized in combination with a compound which has Raf antagonist activity. The instant compounds may also be co-administered with compounds that are selective inhibitors of farnesyl-protein transferase, dual inhibitors of farnesyl-protein transferase and geranylgeranylprotein transferase type I or selective inhibitors of geranylgeranylprotein transferase type I. Such a selective inhibitor or dual inhibitor may be an inhibitor that is competitive with the binding of the CAAX-containing protein substrate of farnesyl-protein transferase or may be farnesyl pyrophosphate competitive inhibitors.

In particular, the compounds disclosed in the following patents and publications may be useful as farnesyl pyrophosphate-competitive inhibitor component of the instant composition: U.S. Ser. Nos. 08/254,228 and 08/435,047. Those patents and publications are incorporated herein by reference.

In practicing methods of this invention, which comprise administering, simultaneously or sequentially or in any order, two or more of a protein substrate-competitive inhibitor and a prenyl pyrophosphate-competitive inhibitor, such administration can be orally or parenterally, including intravenous, intramuscular, intraperitoneal, subcutaneous, rectal and topical routes of administration. It is preferred that such administration be orally. It is more preferred that such administration be orally and simultaneously. When the protein substrate-competitive inhibitor and a prenyl pyrophosphate-competitive inhibitor are administered sequentially, the administration of each can be by the same method or by different methods.

The instant compounds may also be useful in combination with an integrin antagonist for the treatment of cancer, as described in U.S. Ser. No. 09/055,487, filed Apr. 6, 1998, which is incorporated herein by reference.

As used herein the term an integrin antagonist refers to compounds which selectively antagonize, inhibit or counteract binding of a physiological ligand to an integrin(s) that is involved in the regulation of angiogenisis, or in the growth and invasiveness of tumor cells. In particular, the term refers to compounds which selectively antagonize, inhibit or counteract binding of a physiological ligand to the αvβ3 integrin, which selectively antagonize, inhibit or counteract binding of a physiological ligand to the αvβ5 integrin, which antagonize, inhibit or counteract binding of a physiological ligand to both the αvβ3 integrin and the αvβ5 integrin, or which antagonize, inhibit or counteract the activity of the particular integrin(s) expressed on capillary endothelial cells. The term also refers to antagonists of the αvβ6, αvβ8, α1β1, α2β1, α5β1, α6β1 and α6β4 integrins. The term also refers to antagonists of any combination of αvβ3, αvβ5, αvβ6, αvβ8, α1β1, α2β1, α5β1, α6β1 and α6β4 integrins. The instant compounds may also be useful with other agents that inhibit angiogenisis and thereby inhibit the growth and invasiveness of tumor cells, including, but not limited to angiostatin and endostatin.

Similarly, the instant compounds may be useful in combination with agents that are effective in the treatment and prevention of NF-1, restenosis, polycystic kidney disease, infections of hepatitis delta and related viruses and fungal infections.

If formulated as a fixed dose, such combination products employ the combinations of this invention within the dosage range described below and the other pharmaceutically active agent(s) within its approved dosage range. Combinations of the instant invention may alternatively be used sequentially with known pharmaceutically acceptable agent(s) when a multiple combination formulation is inappropriate.

### EXAMPLES

Examples provided are intended to assist in a further understanding of the invention. Particular materials employed, species and conditions are intended to be further illustrative of the invention and not limiting of the reasonable scope thereof.

### Example 1

Preparation of 1-(2-hydroxy-5-methylbenzoyl)-4-[1-(3-((3-(2-hydroxyethoxy) phenyl)oxy)-4-cyanobenzyl)-2-methyl-5-imidazolylmethyl]piperazine (1), dihydrochloride

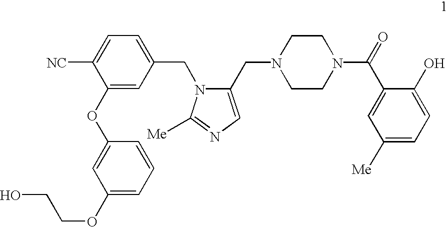

Step A: Preparation of 1-triphenylmethyl-4-(hydroxymethyl)-imidazole

To a solution of 4-(hydroxymethyl)imidazole hydrochloride (35.0 g, 260 mmol) in 250 mL of dry DMF at room temperature was added triethylamine (90.6 mL, 650 mmol). A white solid precipitated from the solution. Chlorotriphenylmethane (76.1 g, 273 mmol) in 500 mL of DMF was added dropwise. The reaction mixture was stirred for 20 hours, poured over ice, filtered, and washed with ice water. The resulting product was slurried with cold dioxane, filtered, and dried in vacuo to provide the titled product as a white solid which was sufficiently pure for use in the next step.

Step B: Preparation of 1-triphenylmethyl-4-(acetoxymethyl)-imidazole

Alcohol from Step A (260 mmol, prepared above) was suspended in 500 mL of pyridine. Acetic anhydride (74 mL, 780 mmol) was added dropwise, and the reaction was stirred for 48 hours during which it became homogeneous. The solution was poured into 2 L of EtOAc, washed with water (3 x 1 L), 5% aq. HCl soln. (2×1 L), sat. aq. NaHCO₃, and brine, then dried (Na₂SO₄), filtered, and concentrated in vacuo to provide the crude product. The acetate was isolated as a white powder which was sufficiently pure for use in the next reaction.

Step C: Preparation of 4-cyano-3-fluorotoluene

To a degassed solution of 4-bromo-3-fluorotoluene (50.0 g, 264 mmol) in 500 mL of DMF was added Zn(CN)₂ (18.6 g, 159 mmol) and Pd(PPh₃)₄ (6.1 g, 5.3 mmol). The reaction was stirred at 80° C. for 6 hours, then cooled to room temperature. The solution was poured into EtOAc, washed with water, sat. aq. NaHCO₃, and brine, then dried (Na₂SO₄), filtered, and concentrated in vacuo to provide the crude product. Purification by silica gel chromatography (0-5% EtOAc/hexane) provided the titled product.

Step D: Preparation of 4-cyano-3-fluorobenzylbromide

To a solution of the product from Step C (22.2 g, 165 mmol) in 220 mL of carbontetrachloride was added N-bromosuccinimide (29.2 g, 164 mmol) and benzoylperoxide (1.1 g). The reaction was heated to reflux for 30 minutes, then cooled to room temperature. The solution was concentrated in vacuo to one-third the original volume, poured into EtOAc, washed with water, sat. aq. NaHCO₃, and brine, then dried (Na₂SO₄), filtered, and concentrated in vacuo to provide the crude product. Analysis by ¹H NMR indicated only partial conversion, so the crude material was resubjected to the same reaction conditions for 2.5 hours, using 18 g (102 mmol) of N-bromosuccinimide. After workup, the crude material was purified by silica gel chromatography (0-10% EtOAc/hexane) to provide the desired product.

Step E: Preparation of 1-(4-cyano-3-fluorobenzyl)-2-methyl-5-imidazolecarboxaldehyde

To a solution of the bromide from Step D (1.26 g, 5.9 mmol) in 10 mL of DMF at 0° C. was added 4-formyl-2-methylimidazole (0.650 g, 5.9 mmol) and cesium carbonate (2.9 g, 8.9 mmol). After 2 hours, the reaction was poured into 2:1 EtOAc:hexane, washed with water and brine, dried (Na₂SO₄), filtered, and concentrated in vacuo to provide the crude product mixture. The material was purified by silica gel chromatography (2-5% MeOH/CHCl3) to provide the titled product along with the regioisomer 1-(4-cyano-3-fluorobenzyl)-2-methyl-4-imidazolecarboxaldehyde and a mixed fraction.

Step F: Preparation of 1-(tert-butoxycarbonyl)-4-[1-(4-cyano-3-fluorobenzyl)-2-methyl-5 -imidazolytmethyl]piperazine

To a solution of 1-tert-butylpiperazine carboxylate (4.02 g, 21.6 mmol) and the aldehyde from Step E (5.0 g, 20.6 mmol) in 50 mL of 1,2-dichloroethane at 0° C. was added 4 Å powdered molecular sieves (2 g), followed by sodium triacetoxyborohydride (6.55 g, 30.9 mmol) and acetic acid (4.6 mL, 82 mmol). The reaction was warmed to room temperature and stirred for 4 hours. The solution was poured into EtOAc, washed with dilute aq. NaHCO₃ and brine, dried (Na₂SO₄), filtered, and concentrated in vacuo. The resulting product was taken up in CH₂Cl₂, and propylamine was added. The mixture was stirred for 30 minutes, then concentrated in vacuo. This material was purified by silica gel chromatography (50-80% acetone/CH₂Cl₂) to give the titled product.

Step G: Preparation 1-(tert-butoxycarbonyl)-4-[1-(3-((3-(2-hydroxyethoxy) phenyl)oxy)-4-cyanobenzyl)-2-methyl-5-imidazolylmethyl]piperazine

To a solution of the product from Step F (1.14 g, 2.77 mmol) and O-(2-hydroxyethyl)resorcinol (1.28 g, 8.31 mmol) in 25 mL of DMSO was added cesium carbonate (2.71 g, 8.31 mmol). The reaction was stirred at room temperature over-night. The solution was poured into EtOAc and washed with water. The aqueous phase was extracted several times with EtOAc. The combined organic layers were washed with brine, dried (Na₂SO₄), filtered, and concentrated in vacuo. The resulting product was purified by silica gel chromatography (5-10% MeOH/EtOAc) to give the titled product as a white solid. This intermediate was active in the assays disclosed below.

Step H: Preparation 4-[1 -(3-((3-(2-hydroxyethoxy)phenyl)oxy)-4-cyanobenzyl)-2-methyl-5-imidazolylmethyl]piperazine hydrochloride

Through a solution of the product from Step I (777 mg, 1.25 mmol) in 10 mL of ethyl acetate at 0C was bubbled anhydrous HCl gas for 4 minutes. After 30 minutes, the solution was concentrated in vacuo to provide the titled salt which was used in the next reaction without further purification.

Step I: Preparation of Compound 1 dihydrochloride

To a solution of the product from Step H (150 mg, 0.28 mmol) and 5-methylsalicylic acid (52 mg, 0.34 mmol), and 1-hydroxybenzotriazole hydrate (46 mg, 0.34 mmol) in 5 mL of dimethylformamide was added 1-(dimethylaminopropyl)-3-ethylcarbodiimide hydrochloride (66 mg, 0.34 mmol) and triethylamine (0.156 mL, 1.12 mmol). After stirring overnight, the reaction was poured into EtOAc and washed with saturated NaHCO₃ solution and brine, and concentrated in vacuo. The resulting material was taken up in 10 mL of methanol, excess potassium carbonate was added, and the solution stirred at room temperature for 2.5 hours. The solution was decanted away from the solids, dried with Na₂SO₄, filtered, and concentrated in vacuo to yield the product as a white foam. A portion of this was taken up in CH₂Cl₂ and treated with excess 1 M HCl/ether solution, and concentrated in vacuo to provide the titled product dihydrochloride as a white powder.

ES mass spectrum m/e 582.4 (M+1).

### Example 2

Preparation of 1-(2-methoxy-5-methylbenzoyl)-4-[1-(3-((3-(2-hydroxyethoxy)phenyl) oxy)-4-cyanobenzyl)-2-methyl-5-imidazolylmethyl]piperazine (2), dihydrochloride

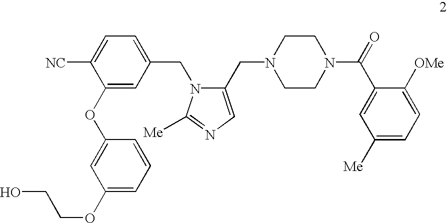

To a solution of the product from Step I of Example 1 (71 mg, 0.12 mmol) in 4 mL of methanol at room temperature was added rimethylsilyl-diazomethane solution (2.0 mL, 2.0 mmol, 2 M in hexane). After stirring overnight, the solution was poured into EtOAc, washed with dilute aq. NaHCO₃ and brine, dried (Na₂SO₄), filtered, and concentrated in vacuo. The resulting product was purified on a 1 mm silica gel preparative TLC plate (90:10:1 CHCl3/MeOH/NH40H), taken up in CH₂Cl₂ and treated with excess 1 M HCl/ether solution, and concentrated in vacuo to provide the titled product dihydrochloride as a white powder.

ES mass spectrum m/e 596.4 (M+1).

### Example 3

Preparation of 1-(2-butoxy-5-methylbenzoyl)-4-[1-(3-((3-(2-hydroxyethoxy)phenyl) oxy)-4-cyanobenzyl)-2-methyl-5-imidazolylmethyl]piperazine (3), dihydrochloride

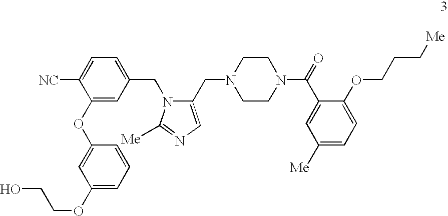

To a solution of the product from Step I of Example 1 (72 mg, 0.12 mmol) in 3 mL of dimethylformamide at room temperature was added cesium carbonate (44 mg, 0.13 mmol) and iodobutane (0.015 mL, 0.13 mmol). After stirring overnight, an additional portion of iodobutane was added (0.009 mL, 0.08 mmol), and the reaction was stirred for 3 hours. The solution was poured into EtOAc, washed with dilute aq. NaHCO₃ and brine, dried (Na₂SO₄), filtered, and concentrated in vacuo. The resulting product was purified on a 1 mm silica gel preparative TLC plate (90:10:1 CHCl₃/MeOH/NH₄OH), taken up in CH₂Cl₂ and treated with excess 1 M HCl/ether solution, and concentrated in vacuo to provide the titled product dihydrochloride as a white powder.

ES mass spectrum m/e 638.5 (M+1).

### Example 4

Preparation of 1-(2-methoxybenzoyl)-4-[1-(3-((3-(2-hydroxyethoxy)phenyl)oxy)-4-cyanobenzyl)-2-methyl-5-imidazolylmethyl]piperazine (4), dihydrochloride

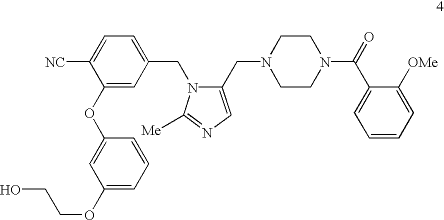

The titled product was prepared using the procedures described in Examples 1 and 2, except that in Step I of Example 1 salicylic acid was used in place of 5-methylsalicylic acid.

### Example 5

Preparation of 1-(2-butoxybenzoyl)-4-[1-(3-((3-(2-hydroxyethoxy)phenyl)oxy)-4-cyanobenzyl)-2-methyl-5-imidazolylmethyl]piperazine (5), dihydrochloride

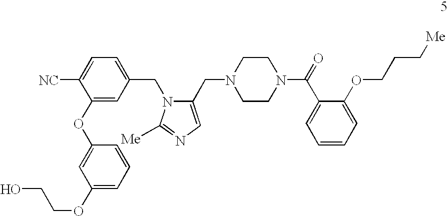

The titled product was prepared using the procedures described in Examples 1 and 3, except that in Step I of Example 1 salicylic acid was used in place of 5-methylsalicylic acid.

ES mass spectrum m/e 624.5 (M+1).

### Example 6

Preparation of 1-(2-methoxy-4-methylbenzoyl)-4-[1-(3-((3-(2-hydroxyethoxy) phenyl)oxy)-4-cyanobenzyl)-2-methyl-5-imidazolylmethyl]piperazine (6), dihydrochloride

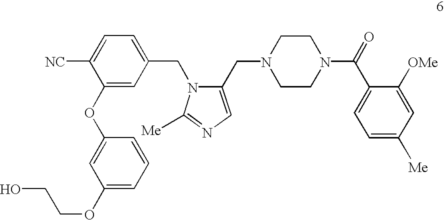

The titled product was prepared using the procedures described in Examples 1 and 2, except that in Step I of Example 14-methylsalicylic acid was used in place of 5-methylsalicylic acid.

ES mass spectrum m/e 596.4 (M+1).

### Example 7

Preparation of 1-(2-butoxy-4-methylbenzoyl)-4-[1-(3-((3-(2-hydroxyethoxy)phenyl) oxy)-4-cyanobenzyl)-2-methyl-5-imidazolylmethyl]piperazine (7), dihydrochloride

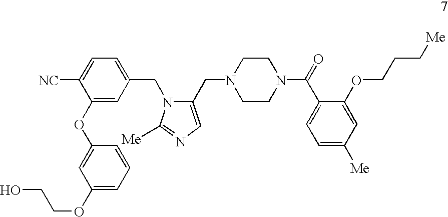

The titled product was prepared using the procedures described in Examples 1 and 3, except that in Step I of Example 14-methylsalicylic acid was used in place of 5-methylsalicylic acid.

ES mass spectrum m/e 638.5 (M+1).

### Example 8

Preparation of 1-(2-methoxy-3-methylbenzoyl)-4-[1-(3-((3-(2-hydroxyethoxy)phenyl) oxy)-4-cyanobenzyl)-2-methyl-5-imidazolylmethyl]piperazine (8), dihydrochloride

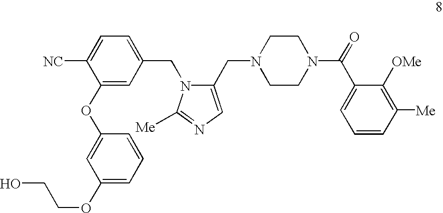

The titled product was prepared using the procedures described in Examples 1 and 2, except that in Step I of Example 13-methylsalicylic acid was used in place of 5-methylsalicylic acid.

ES mass spectrum m/e 596.4 (M+1).

### Example 9

Preparation of 1-(2-butoxy-3-methylbenzoyl)-4-[1-(3-((3-(2-hydroxyethoxy)phenyl) oxy)-4-cyanobenzyl)-2-methyl-5-imidazolylmethyl]piperazine (9), dihydrochloride

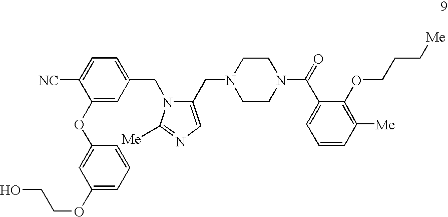

The titled product was prepared using the procedures described in Examples 1 and 3, except that in Step I of Example 13-methylsalicylic acid was used in place of 5-methylsalicylic acid.

ES mass spectrum m/e 638.5 (M+1).

### Example 10

In vitro Inhibition of Ras Farnesyl Transferase

Transferase Assays. Isoprenyl-protein transferase activity assays are carried out at 30° C. unless noted otherwise. A typical reaction contains (in a final volume of 50 μL): [³H]farnesyl diphosphate, Ras protein, 50 mM HEPES, pH 7.5, 5 mM MgCl₂, 5 mM dithiothreitol, 10 μM ZnCl₂, 0.1% polyethyleneglycol (PEG) (15,000-20,000 mw) and isoprenyl-protein transferase. The FPTase employed in the assay is prepared by recombinant expression as described in Omer, C. A., Kral, A. M., Diehl, R. E., Prendergast, G. C., Powers, S., Allen, C. M., Gibbs, J. B. and Kohl, N. E. (1993) Biochemistry 32:5167-5176. After thermally pre-equilibrating the assay mixture in the absence of enzyme, reactions are initiated by the addition of isoprenyl-protein transferase and stopped at timed intervals (typically 15 min) by the addition of 1 M HCl in ethanol (1 mL). The quenched reactions are allowed to stand for 15 m (to complete the precipitation process). After adding 2 mL of 100% ethanol, the reactions are vacuum-filtered through Whatman GF/C filters. Filters are washed four times with 2 mL aliquots of 100% ethanol, mixed with scintillation fluid (10 mL) and then counted in a Beckman LS3801 scintillation counter.

For inhibition studies, assays are run as described above, except inhibitors are prepared as concentrated solutions in 100% dimethyl sulfoxide and then diluted 20-fold into the enzyme assay mixture. Substrate concentrations for inhibitor IC₅₀ determinations are as follows: FTase, 650 nM Ras-CVLS (SEQ.ID.NO.: 1), 100 nM farnesyl diphosphate.

The compounds of the instant invention described in the above Examples 1-9 were tested for inhibitory activity against human FPTase by the assay described above and were found to have an IC50 of ≦5 μM.

### Example 11

Modified In vitro GGTase Inhibition Assay

The modified geranylgeranyl-protein transferase inhibition assay is carried out at room temperature. A typical reaction contains (in a final volume of 50 μL): [³H]geranylgeranyl diphosphate, biotinylated Ras peptide, 50 MM HEPES, pH 7.5, a modulating anion (for example 10 mM glycerophosphate or 5mM ATP), 5 mM MgCl₂, 10 μM ZnCI2, 0.1% PEG (15,000-20,000 mw), 2 mM dithiothreitol, and geranylgeranyl-protein transferase type I(GGTase). The GGTase-type I enzyme employed in the assay is prepared as described in U.S. Pat. No. 5,470,832, incorporated by reference. The Ras peptide is derived from the K4B-Ras protein and has the following sequence: biotinyl-GKKKKKKSKTKCVIM (single amino acid code) (SEQ.ID.NO.: 2). Reactions are initiated by the addition of GGTase and stopped at timed intervals (typically 15 min) by the addition of 200 μL of a 3 mg/mL suspension of streptavidin SPA beads (Scintillation Proximity Assay beads, Amersham) in 0.2 M sodium phosphate, pH 4, containing 50 mM EDTA, and 0.5% BSA. The quenched reactions are allowed to stand for 2 hours before analysis on a Packard TopCount scintillation counter.

For inhibition studies, assays are run as described above, except inhibitors are prepared as concentrated solutions in 100% dimethyl sulfoxide and then diluted 25-fold into the enzyme assay mixture. IC50 values are determined with Ras peptide near K_{M} concentrations. Enzyme and substrate concentrations for inhibitor IC₅₀ determinations are as follows: 75 pM GGTase-I, 1.6 μM Ras peptide, 100 nM geranylgeranyl diphosphate.

The compounds of the instant invention described in the above Examples 1-9 were tested for inhibitory activity against human GGTase-type I by the assay described above and were found to have an IC₅₀ of ≦5 μM.

### Example 12

Cell-based In vitro Ras Farnesylation Assay

The cell line used in this assay is a v-ras line derived from either Rat1 or NIH3T3 cells, which expressed viral Ha-ras p21. The assay is performed essentially as described in DeClue, J. E. et al., Cancer Research 51:712-717, (1991). Cells in 10 cm dishes at 50-75% confluency are treated with the test compound (final concentration of solvent, methanol or dimethyl sulfoxide, is 0.1%). After 4 hours at 37° C., the cells are labeled in 3 mL methionine-free DMEM supple-mented with 10% regular DMEM, 2% fetal bovine serum and 400 μCi[³⁵S]methionine (1000 Ci/mmol). After an additional 20 hours, the cells are lysed in 1 mL lysis buffer (1% NP40/20 mM HEPES, pH 7.5/5 mM MgCl₂/1 mM DTT/10 mg/mL aprotinen/2 mg/mL leupeptin/2 mg/mL antipain/0.5 mM PMSF) and the lysates cleared by centrifugation at 100,000×g for 45 min. Aliquots of lysates containing equal numbers of acid-precipitable counts are bought to 1 mL with IP buffer (lysis buffer lacking DTT) and immunoprecipitated with the ras-specific monoclonal antibody Y13-259 (Furth, M. E. et al., J. Virol. 43:294-304, (1982)). Following a 2 hour antibody incubation at 4° C., 200 μl of a 25% suspension of protein A-Sepharose coated with rabbit anti rat IgG is added for 45 min. The immunoprecipitates are washed four times with IP buffer (20 nM HEPES, pH 7.5/1 mM EDTA/1 % Triton X-100.0.5% deoxycholate/0.1%/SDS/0.1 M NaCl) boiled in SDS-PAGE sample buffer and loaded on 13% acrylamide gels. When the dye front reached the bottom, the gel is fixed, soaked in Enlightening, dried and autoradiographed. The intensities of the bands corresponding to farnesylated and nonfarnesylated ras proteins are compared to determine the percent inhibition of farnesyl transfer to protein.

### Example 13

Cell-based In vitro Growth Inhibition Assay

To determine the biological consequences of FPTase inhibition, the effect of the compounds of the instant invention on the anchorage-independent growth of Rat1 cells transformed with either a v-ras, v-raf, or v-mos oncogene is tested. Cells transformed by v-Raf and v-Mos maybe included in the analysis to evaluate the specificity of instant compounds for Ras-induced cell transformation.

Rat 1 cells transformed with either v-ras, v-raf, or v-mos are seeded at a density of 1×10⁴ cells per plate (35 mm in diameter) in a 0.3% top agarose layer in medium A (Dulbecco's modified Eagle's medium supplemented with 10% fetal bovine serum) over abottom agarose layer (0.6%). Both layers contain 0.1% methanol or an appropriate concentration of the instant compound (dissolved in methanol at 1000 times the final concentration used in the assay). The cells are fed twice weekly with 0.5 mL of medium A containing 0.1% methanol or the concentration of the instant compound. Photomicrographs are taken 16 days after the cultures are seeded and comparisons are made.

### Example 14

Construction of SEAP reporter plasmid pDSE10

The SEAP reporter plasmid, pDSE100 was constructed by ligating a restriction fragment containing the SEAP coding sequence into the plasmid pCMV-RE-AKI. The SEAP gene is derived from the plasmid pSEAP2-Basic (Clontech, Palo Alto, Calif.). The plasmid pCMV-RE-AKI was constructed by Deborah Jones (Merck) and contains 5 sequential copies of the ‘dyad symmetry response element’ cloned upstream of a ‘CAT-TATA’ sequence derived from the cytomegalo-virus immediate early promoter. The plasmid also contains a bovine growth hormone poly-A sequence.

The plasmid, pDSE100 was constructed as follows. A restriction fragment encoding the SEAP coding sequence was cut out of the plasmid pSEAP2-Basic using the restriction enzymes EcoR1 and HpaI. The ends of the linear DNA fragments were filled in with the Klenow fragment of E. coli DNA Polymerase I. The ‘blunt ended’ DNA containing the SEAP gene was isolated by electrophoresing the digest in an agarose gel and cutting out the 1694 base pair fragment. The vector plasmid pCMV-RE-AKI was linearized with the restriction enzyme Bgl-II and the ends filled in with Klenow DNA Polymerase I. The SEAP DNA fragment was blunt end ligated into the pCMV-RE-AKI vector and the ligation products were transformed into DH5-alpha E. Coli cells (Gibco-BRL). Transformants were screened for the proper insert and then mapped for restriction fragment orientation. Properly oriented recombinant constructs were sequenced across the cloning junctions to verify the correct sequence. The resulting plasmid contains the SEAP coding sequence downstream of the DSE and CAT-TATA promoter elements and upstream of the BGH poly-A sequence.

Alternative Construction of SEAP reporter plasmid, pDSE101

The SEAP repotrer plasmid, pDSE101 is also constructed by ligating a restriction fragment containing the SEAP coding sequence into the plasmid pCMV-RE-AKI. The SEAP gene is derived from plasmid pGEM7zf(-)/SEAP.

The plasmid pDSE11 was constructed as follows: A restriction fragment containing part of the SEAP gene coding sequence was cut out of the plasmid pGEM7zf(-)/SEAP using the restriction enzymes Apa I and KpnI. The ends of the linear DNA fragments were chewed back with the Klenow fragment of E. coli DNA Polymerase I. The “blunt ended” DNA containing the truncated SEAP gene was isolated by electrophoresing the digest in an agarose gel and cutting out the 1910 base pair fragment. This 1910 base pair fragment was ligated into the plasmid pCMV-RE-AKI which had been cut with Bgl-II and filled in with E. coli Klenow fragment DNA polymerase. Recombinant plasmids were screened for insert orientation and sequenced through the ligated junctions. The plasmid pCMV-RE-AKI is derived from plasmid pCMVIE-AKI-DHFR (Whang, Y., Silberklang, M., Morgan, A., Munshi, S., Lenny, A. B., Ellis, R. W., and Kieff, E. (1987) J. Virol., 61, 1796-1807) by removing an EcoRI fragment containing the DHFR and Neomycin markers. Five copies of the fos promoter serum response element were inserted as described previously (Jones, R. E., Defeo-Jones, D., McAvoy, E. M., Vuocolo, G. A., Wegrzyn, R. J., Haskell, K. M. and Oliff, A. (1991) Oncogene, 6, 745-751) to create plasmid pCMV-RE-AKI.

The plasmid pGEM7zf(-)/SEAP was constructed as follows. The SEAP gene was PCRed, in two segments from a human placenta cDNA library (Clontech) using the following oligos.

| | |
|---|---|
| Antisense strand N-terminal SEAP: | |
| 5′ GAGAGGGAATTCGGGCCCTTCCTGCATGCTGCTGCTGCTGGGC | (SEQ.ID.NO.:3) |
| Antisense strand N-terminal SEAP: | |
| 5′ GAGAGAGCTCGAGGTTAACCCGGGTGCGCGGCGTCGGTGGT 3′ | (SEQ.ID.NO.:4) |
| Sense strand C-terminal SEAP: | |
| 5′ GAGAGAGTCTAGAGTTAACCCGTGGTCCCCGCGTTGCTTCCT 3′ | (SEQ.ID.NO.:5) |
| Antisense strand C-terminal SEAP: | |
| 5′ GAAGAGGAAGCTTGGTACCGCCACTGGGCTGTAGGTGGTGGCT 3′ | (SEQ.ID.NO.:6) |

The N-terminal oligos (SEQ.ID.NO.: 4 and SEQ.ID.NO.: 5) were used to generate a 1560 bp N-terminal PCR product that contained EcoRI and HpaI restriction sites at the ends. The Antisense N-terminal oligo (SEQ.ID.NO.: 4) introduces an internal translation STOP codon within the SEAP gene along with the HpaI site. The C-terminal oligos (SEQ.ID.NO.: 5 and SEQ.ID.NO.: 6) were used to amplify a 412 bp C-terminal PCR product containing HpaI and HindII restriction sites. The sense strand C-terminal oligo (SEQ.ID.NO.: 5) introduces the internal STOP codon as well as the HpaI site. Next, the N-terminal amplicon was digested with EcoRI and HpaI while the C-terminal amplicon was digested with HpaI and HindIII. The two fragments comprising each end of the SEAP gene were isolated by electrophoresing the digest in an agarose gel and isolating the 1560 and 412 base pair fragments. These two fragments were then co-ligated into the vector pGEM7zf(-) (Promega) which had been restriction digested with EcoRI and HindIII and isolated on an agarose gel. The resulting clone, pGEM7zf(-)/SEAP contains the coding sequence for the SEAP gene from amino acids.

Construction of a Constitutively Expressing SEAP Plasmid pCMV-SEAP

An expression plasmid constitutively expressing the SEAP protein was created by placing the sequence encoding a truncated SEAP gene downstream of the cytomegalovirus (CMV) IE-1 promoter. The expression plasmid also includes the CMV intron A region 5′ to the SEAP gene as well as the 3′ untranslated region of the bovine growth hormone gene 3′ to the SEAP gene.

The plasmid pCMVIE-AKI-DHFR (Whang et al, 1987) containing the CMV immediate early promoter was cut with EcoRI generating two fragments. The vector fragment was isolated by agarose electrophoresis and religated. The resulting plasmid is named pCMV-AKI. Next, the cytomegalovirus intron A nucleotide sequence was inserted downstream of the CMV IE1 promter in pCMV-AKI. The intron A sequence was isolated from a genomic clone bank and subcloned into pBR322 to generate plasmid p16T-286. The intron A sequence was mutated at nucleotide 1856 (nucleotide numbering as in Chapman, B. S., Thayer, R. M., Vincent, K. A. and Haigwood, N. L., Nuc.Acids Res. 19, 3979-3986) to remove a Sacd restriction site using site directed mutagenesis. The mutated intron A sequence was PCRed from the plasmid pl6T-287 using the following oligos.

| | |
|---|---|
| Sense strand: | |
| 5′ GGCAGAGCTCGTTTAGTGAACCGTCAG 3′ | (SEQ.ID.NO.:7) |
| Antisense strand: | |
| 5′ GAGAGATCTCAAGGACGGTGACTGCAG 3′ | (SEQ.ID.NO.:8) |

These two oligos generate a 991 base pair fragment with a SacI site incorporated by the sense oligo and a Bgl-II fragment incorporated by the antisense oligo. The PCR fragment is trimmed with SacI and Bgl-II and isolated on an agarose gel. The vector pCMV-AKI is cut with SacI and Bgl-II and the larger vector fragment isolated by agarose gel electrophoresis. The two gel isolated fragments are ligated at their respective Sacd and Bgl-ll sites to create plasmid pCMV-AKI-InA.

The DNA sequence encoding the truncated SEAP gene is inserted into the pCMV-AKI-InA plasmid at the Bgl-II site of the vector. The SEAP gene is cut out of plasmid pGEM7zf(-)/SEAP (described above) using EcoRI and HindII. The fragment is filled in with Klenow DNA polymerase and the 1970 base pair fragment isolated from the vector fragment by agarose gel electrophoresis. The pCMV-AKI-hiA vector is prepared by digesting with Bgl-II and filling in the ends with Klenow DNA polymerase. The final construct is generated by blunt end ligating the SEAP fragment into the pCMV-AKI-inA vector. Transformants were screened for the proper insert and then mapped for restriction fragment orientation. Properly oriented recombinant constructs were sequenced across the cloning junctions to verify the correct sequence. The resulting plasmid, named pCMV-SEAP, contains a modified SEAP sequence downstream of the cytomegalovirus immediately early promoter IE-1 and intron A sequence and upstream of the bovine growth hormone poly-A sequence. The plasmid expresses SEAP in a constitutive manner when transfected into mammalian cells.

Cloning of a Myristylated viral-H-ras expression plasmid

A DNA fragment containing viral-H-ras can be PCRed from plasmid “H-1” (Ellis R. et al. J. Virol. 36, 408, 1980) or “HB-11(deposited in the ATCC under Budapest Treaty on Aug. 27, 1997, and designated ATCC_{209,218}) using the following oligos.

| | |
|---|---|
| Sense strand: | |
| 5′TCTCCTCGAGGCCACCATGGGGAGTAGCAAGAGCAAGCCTAAGGACCC | (SEQ.ID.NO.:9) |
| CAGCCAGCGCCGGATGACAGAATACAAGCTTGTGGTGG 3′. | |

| | |
|---|---|
| Antisense: | |
| 5′CACATCTAGATCAGGACAGCACAGACTTGCAGC 3′. | (SEQ.ID. |
| | NO.:10) |

A sequence encoding the first 15 aminoacids of the v-src gene, containing a myristylation site, is incorporated into the sense strand oligo. The sense strand oligo also optimizes the ‘Kozak’ translation initiation sequence immediately 5′ to the ATG start site. To prevent prenylation at the viral-ras C-terminus, cysteine 186 would be mutated to a serine by substituting a G residue for a C residue in the C-terminal antisense oligo. The PCR primer oligos introduce an XhoI site at the 5′ end and a XbaI site at the 3′ end. The XhoI-XbaI fragment can be ligated into the mammalian expression plasmid pCI (Promega) cut with XhoI and XbaI. This results in a plasmid in which the recombinant myr-viral-H-ras gene is constitutively transcribed from the CMV promoter of the pCI vector.

Cloning of a viral-H-ras-CVLL expression plasmid

A viral-H-ras clone with a C-terminal sequence encoding the amino acids CVLL can be cloned from the plasmid “H-1” (Ellis R. et al. J. Virol. 36, 408, 1980) or “HB-11 (deposited in the ATCC under Budapest Treaty on Aug. 27, 1997, and designated ATCC_{209,218}) by PCR using the following oligos.

| | |
|---|---|
| Sense strand: | |
| 5′TCTCCTCGAGGCCACCATGACAGAATACAAGCTTGTGGTGG-3′ | (SEQ.ID.NO.:11) |

| | |
|---|---|
| Antisense strand: | |
| 5′CACTCTAGACTGGTGTCAGAGCAGCACACACTTGCAGC-3′ | (SEQ.ID.NO.:12) |

The sense strand oligo optimizes the ‘Kozak’ sequence and adds an XhoI site. The antisense strand mutates serine 189 to leucine and adds an XbaI site. The PCR fragment can be trimmed with XhoI and XbaI and ligated into the XhoI-XbaI cut vector pCI (Promega). This results in a plasmid in which the mutated viral-H-ras-CVLL gene is constitutively transcribed from the CMV promoter of the pCI vector.

Cloning of c-H-ras-Leu61 Expression Plasmid

The human c-H-ras gene can be PCRed from a human cerebral cortex cDNA library (Clontech) using the following oligonucleotide primers.

| | |
|---|---|
| Sense strand: | |
| 5′-GAGAGAATTCGCCACCATGACGGAATATAAGCTGGTGG-3′ | (SEQ.ID.NO.:13) |

| | |
|---|---|
| Antisense strand: | |
| 5′-GAGAGTCGACGCGTCAGGAGAGCACACACTTGC-3′ | (SEQ.ID. |
| | NO.:14) |

The primers will amplify a c-H-ras encoding DNA fragment with the primers contributing an optimized ‘Kozak’ translation start sequence, an EcoRI site at the N-terminus and a Sal I site at the C-terminal end. After trimming the ends of the PCR product with EcoRI and Sal I, the c-H-ras fragment can be ligated ligated into an EcoRI-Sal I cut mutagenesis vector pAlter-I (Promega). Mutation of glutamine-61 to a leucine can be accomplished using the manufacturer's protocols and the following oligonucleotide:

| | |
|---|---|
| 5′-CCGCCGGCCTGGAGGAGTACAG-3′ | (SEQ.ID.NO.:15) |

After selection and sequencing for the correct nucleotide substitution, the mutated c-H-ras-Leu61l can be excised from the pAlter-1 vector, using EcoRI and Sal I, and be directly ligated into the vector pCI (Promega) which has been digested with EcoRI and Sal I. The new recombinant plasmid will constitutively transcribe c-H-ras-Leu6l from the CMV promoter of the pCI vector.

Cloning of a c-N-ras-Val-12 Expression Plasmid

The human c-N-ras gene can be PCRed from a human cerebral cortex cDNA library (Clontech) using the following oligonucleotide primers.

| | |
|---|---|
| Sense strand: | |
| 5′-GAGAGAATTCGCCACCATGACTGAGTACAAACTGGTGG-3′ | (SEQ.ID.NO.:16) |

| | |
|---|---|
| Antisense strand: | |
| 5′-GAGAGTCGACTTGTTACATCACCACACATGGC-3′ | (SEQ.ID. |
| | NO.:17) |

The primers will amplify a c-N-ras encoding DNA fragment with the primers contributing an optimized ‘Kozak’ translation start sequence, an EcoRI site at the N-terminus and a Sal I stite at the C-terminal end. After trimming the ends of the PCR product with EcoR1 and Sal I, the c-N-ras fragment can be ligated into an EcoRI-Sal I cut mutagenesis vector pAlter-1 (Promega). Mutation of glycine-12 to a valine can be accomplished using the manufacturer's protocols and the following oligonucleotide:

| | |
|---|---|
| 5′-GTTGGAGCAGTTGGTGTTGGG-3′ | (SEQ.ID.NO.:18) |

After selection and sequencing for the correct nucleotide substitution, the mutated c-N-ras-Val-12 can be excised from the pAlter-1 vector, using EcoRI and Sal I, and be directly ligated into the vector pCI (Promega) which has been digested with EcoRI and Sal I. The new recombinant plasmid will constitutively transcribe c-N-ras-Val-12 from the CMV promoter of the pCI vector.

Cloning of a c-K-ras-Val-12 Expression Plasmid

The human c-K-ras gene can be PCRed from a human cerebral cortex cDNA library (Clontech) using the following oligonucleotide primers.

| | |
|---|---|
| Sense strand: | |
| 5′-GAGAGGTACCGCCACCATGACTGAATATAAACTTGTGG-3′ | (SEQ.ID.NO.:19) |

| | |
|---|---|
| Antisense strand: | |
| 5′-CTCTGTCGACGTATTTACATAATTACACACTTTGTC-3′ | (SEQ.ID.NO.:20) |

The primers will amplify a c-K-ras encoding DNA fragment with the primers contributing an optimized ‘Kozak’ translation start sequence, a KpnI-site at the N-terminus and a Sal I stite at the C-terminal end. After trimming the ends of the PCR product with Kpn I and Sal I, the c-K-ras fragment can be ligated into a KpnI-Sal I cut mutagenesis vector pAlter-1 (Promega). Mutation of cysteine-12 to a valine can be accomplished using the manufacturer's protocols and the following oligonucleotide:

| | |
|---|---|
| 5′-GTAGTTGGAGCTGTTGGCGTAGGC-3′ | (SEQ.ID.NO.:21) |

After selection and sequencing for the correct nucleotide substitution, the mutated c-K-ras-Val-12 can be excised from the pAlter-1 vector, using KpnI and Sal I, and be directly ligated into the vector pCI (Promega) which has been digested with KpnI and Sal I. The new recombinant plasmid will constitutively transcribe c-K-ras-Val-12 from the CMV promoter of the pCI vector.

SEAP Assay

Human C33A cells (human epitheial carcenoma-ATTC collection) are seeded in 10cm tissue culture plates in DMEM +10% fetal calf serum+1× Pen/Strep+1X glutamine +1X NEAA. Cells are grown at 370C in a 5% CO₂ atmosphere until they reach 50 -80% of confluency.

The transient transfection is performed by the CaPO4 method (Sambrook et al., 1989). Thus, expression plasmids for H-ras, N-ras, K-ras, Myr-ras or H-ras-CVLL are co-precipitated with the DSE-SEAP reporter construct. For 10 cm plates 600μl of CaCl₂ -DNA solution is added dropwise while vortexing to 600μl of 2X HBS buffer to give 1.2ml of precipitate solution (see recipes below). This is allowed to sit at room temperature for 20 to 30 minutes. While the precipitate is forming, the media on the C33A cells is replaced with DMEM (minus phenol red; Gibco cat. # 31053-028)+0.5% charcoal stripped calf serum +IX (Pen/Strep, Glutamine and nonessential aminoacids). The CaPO4-DNA precipitate is added dropwise to the cells and the plate rocked gently to distribute. DNA uptake is allowed to proceed for 5-6 hrs at 370C under a 5% C02 atmosphere.

Following the DNA incubation period, the cells are washed with PBS and trypsinized with 1 ml of 0.05% trypsin. The 1 mL of trypsinized cells is diluted into 10 ml of phenol red free DMEM +0.2% charcoal stripped calf serum +1X (Pen/Strep, Glutamine and NEAA). Transfected cells are plated in a 96 well microtiter plate (100μl/well) to which drug, diluted in media, has already been added in a volume of 100μl. The final volume per well is 200 μl with each drug concentration repeated in triplicate over a range of half-log steps.

Incubation of cells and drugs is for 36 hrs at 37° C. under C02. At the end of the incubation period, cells are examined microscopically for evidence of cell distress. Next, 100 μl of media containing the secreted alkaline phosphatase is removed from each well and transferred to a microtube array for heat treatment at 65° C. for 1 hr to inactivate endogenous alkaline phosphatases (but not the heat stable secreted phosphatase).

The heat treated media is assayed for alkaline phosphatase by a luminescence assay using the luminescence reagent CSPD(® (Tropix, Bedford, Mass.). A volume of 50 μl media is combined with 200 μl of CSPD cocktail and incubated for 60 minutes at room temperature. Luminesence is monitored using an ML2200 microplate luminometer (Dynatech). Luminescence reflects the level of activation of the fos reporter construct stimulated by the transiently expressed protein.

| | | |
|---|---|---|
| DNA-CaPO₄ precipitate for 10 cm. plate of cells | | |
| Ras expression plasmid (1 μg/μl) | 10 | μl |
| DSE-SEAP Plasmid (1 μg/μl) | 2 | μl |
| Sheared Calf Thymus DNA (1 μg/μl) | 8 | μl |
| 2 M CaCl₂ | 74 | μl |
| dH₂O | 506 | μl |
| 2X HBS Buffer | | |
| 280 mM NaCl | | |
| 10 mM KCl | | |
| 1.5 mM Na₂HPO₄ 2H₂O | | |
| 12 mM dextrose | | |
| 50 mM HEPES | | |
| Final pH = 7.05 | | |
| Luminesence Buffer (26 ml) | | |
| Assay Buffer | 20 | ml |
| Emerald Reagent ™ (Tropix) | 2.5 | ml |
| 100 mM homoarginine | 2.5 | ml |
| CSPD Reagent ® (Tropix) | 1.0 | ml |
| Assay Buffer | | |
| Add 0.05 M Na₂CO₃ to 0.05 M NaHCO₃ | | |
| to obtain pH 9.5. | | |
| Make 1 mM in MgCl₂ | | |

### Example 15

The processing assays employed are modifications of that described by DeClue et al [Cancer Research 51, 712-717 1991]

K4B-Ras Processing Inhibition Assay

PSN-1 (human pancreatic carcinoma) or viral-K4B-ras-transformed RatI cells are used for analysis of protein processing. Subconfluent cells in 100 mm dishes are fed with 3.5 mL of media (methionine-free RPMI supplemented with 2% fetal bovine serum or cysteine-free/methionine-free DMEM supplemented with 0.035 mL of 200 mM glutamine (Gibco), 2% fetal bovine serum, respectively) containing the desired concentration of test compound, lovastatin or solvent alone. Cells treated with lovastatin (5-10 EM), a compound that blocks Ras processing in cells by inhibiting a rate-limiting step in the isoprenoid biosynthetic pathway, serve as a positive control. Test compounds are prepared as 1000× concentrated solutions in DMSO to yield a final solvent concentration of 0.1%. Following incubation at 37° C. for two hours 204 μCi/mL [³⁵S]Pro-Mix (Amersham, cell labeling grade) is added.

After introducing the label amino acid mixture, the cells are incubated at 37° C. for an additional period of time (typically 6 to 24 hours). The media is then removed and the cells are washed once with cold PBS. The cells are scraped into 1 mL of cold PBS, collected by centrifugation (10,000×g for 10 sec at room temperature), and lysed by vortexing in 1 mL of lysis buffer (1% Nonidet P-40, 20 mM HEPES, pH 7.5, 150 mM NaCl, 1 mM EDTA, 0.5% deoxycholate, 0.1% SDS, -1 mM DTT, 10 μg/mL AEBSF, 10 μg/mL aprotinin, 2 μg/mL leupeptin and 2 μg/mL antipain). The lysate is then centrifuged at 15,000×g for 10 min at 4° C. and the supernatant saved.

For immunoprecipitation of Ki4B-Ras, samples of lysate supernatant containing equal amounts of protein are utilized. Protein concentration is determined by the bradford method utilizing bovine serum albumin as a standard. The appropriate volume of lysate is brought to 1 mL with lysis buffer lacking DTT and 8 μg of the pan Ras monoclonal antibody, Y13-259, added. The protein/antibody mixture is incubated on ice at 4° C. for 24 hours. The immune complex is collected on pansorbin (Calbiochem) coated with rabbit antiserum to rat IgG (Cappel) by tumbling at 4° C. for 45 minutes. The pellet is washed 3 times with 1 mL of lysis buffer lacking DTT and protease inhibitors and resuspended in 100 μl elution buffer (10 mM Tris pH 7.4, 1% SDS). The Ras is eluted from the beads by heating at 95° C. for 5 minutes, after which the beads are pelleted by brief centrifugation (15,000×g for 30 sec. at room temperature).

The supernatant is added to 1 mL of Dilution Buffer 0.1 % Triton X-100, 5 mM EDTA, 50 mM NaCl, 10 mM Tris pH 7.4) with 2 μg Kirsten-ras specific monoclonal antibody, c-K-ras Ab-I (Calbiochem). The second protein/antibody mixture is incubated on ice at 4° C. for 1-2 hours. The immune complex is collected on pansorbin (Calbiochem) coated with rabbit antiserum to rat IgG (Cappel) by tumbling at 4° C. for 45 minutes. The pellet is washed 3 times with 1 mL of lysis buffer lacking DTT and protease inhibitors and resuspended in Laemmli sample buffer. The Ras is eluted from the beads by heating at 95° C. for 5 minutes, after which the beads are pelleted by brief centrifugation. The supernatant is subjected to SDS-PAGE on a 12% acrylamide gel (bis-acrylamide:acrylamide, 1: 100), and the Ras visualized by fluorography.

hDJ Processing Inhibition Assay

PSN-1 cells are seeded in 24-well assay plates. For each compound to be tested, the cells are treated with a minimum of seven concentrations in half-log steps. The final solvent (DMSO) concentration is 0.1%. A vehicle-only control is included on each assay plate. The cells are treated for 24 hours at 37° C. / 5% CO₂.

The growth media is then aspirated and the samples are washed with PBS. The cells are lysed with SDS-PAGE sample buffer containing 5% 2-mercaptoethanol and heated to 95° C. for 5 minutes. After cooling on ice for 10 minutes, a mixture of nucleases is added to reduce viscosity of the samples.

The plates are incubated on ice for another 10 minutes. The samples are loaded onto pre-cast 8% acrylamide gels and electrophoresed at 15 mA/gel for 3-4 hours. The samples are then transferred from the gels to PVDF membranes by Western blotting.

The membranes are blocked for at least 1 hour in buffer containing 2% nonfat dry milk. The membranes are then treated with a monoclonal antibody to hDJ-2 (Neomarkers Cat. # MS-225), washed, and treated with an alkaline phosphatase-conjugated secondary antibody. The membranes are then treated with a fluorescent detection reagent and scanned on a phosphorimager.

For each sample, the percent of total signal corresponding to the unprenylated species of hDJ (the slower-migrating species) is calculated by densitometry. Dose-response curves and EC₅₀ values are generated using 4-parameter curve fits in SigmaPlot software.

### Example 16

Rap1 Processing Inhibition Assay

Protocol A:

Cells are labeled, incubated and lysed as described in Example 15.

For immunoprecipitation of Rap I, samples of lysate supernatant containing equal amounts of protein are utilized. Protein concentration is determined by the bradford method utilizing bovine serum albumin as a standard. The appropriate volume of lysate is brought to 1 mL with lysis buffer lacking DTT and 2 μg of the Rap1 antibody, Rap1/Krev1 (121) (Santa Cruz Biotech), is added. The protein/antibody mixture is incubated on ice at 4° C. for 1 hour. The immune complex is collected on pansorbin (Calbiochem) by tumbling at 4° C. for 45 minutes. The pellet is washed 3 times with 1 mL of lysis buffer lacking DTT and protease inhibitors and resuspended in 100 μl elution buffer (10 mM Tris pH 7.4, 1% SDS). The Rap1 is eluted from the beads by heating at 95° C. for 5 minutes, after which the beads are pelleted by brief centrifugation (15,000×g for 30 sec. at room temperature).

The supernatant is added to 1 mL of Dilution Buffer (0.1 % Triton X-100, 5 mM EDTA, 50 mM NaCl, 10 mM Tris pH 7.4) with 2 μg Rapl antibody, Rap I/Krevl (121) (Santa Cruz Biotech). The second protein/antibody mixture is incubated on ice at 4° C. for 1-2 hours. The immune complex is collected on pansorbin (Calbiochem) by tumbling at 4° C. for 45 minutes. The pellet is washed 3 times with 1 mL of lysis buffer lacking DTT and protease inhibitors and resuspended in Laemmli sample buffer. The Rap1 is eluted from the beads by heating at 95° C. for 5 minutes, after which the beads are pelleted by brief centrifugation. The supernatant is subjected to SDS-PAGE on a 12% acrylamide gel (bis-acrylamide:acrylamide, 1:100), and the Rap1 visualized by fluorography.

Protocol B:

PSN-1 cells are passaged every 3-4 days in 10cm plates, splitting near-confluent plates 1:20 and 1:40. The day before the assay is set up, 5×10⁶ cells are plated on 15 cm plates to ensure the same stage of confluency in each assay. The media for these cells is RPM11640 (Gibco), with 15% fetal bovine serum and lx Pen/Strep antibiotic mix.

The day of the assay, cells are collected from the 15cm plates by trypsinization and diluted to 400,000 cells/mL in media. 0.5ml of these diluted cells are added to each well of 24-well plates, for a final cell number of 200,000 per well. The cells are then grown at 37° C. overnight.

The compounds to be assayed are diluted in DMSO in ½-log dilutions. The range of final concentrations to be assayed is generally 0.1-100 μM. Four concentrations per compound is typical. The compounds are diluted so that each concentration is 1000× of the final concentration (i.e., for a 10 μM data point, a 10 mM stock of the compound is needed).

2 μL of each 1000× compound stock is diluted into 1 ml media to produce a 2X stock of compound. A vehicle control solution (2μL DMSO to Iml media), is utilized. 0.5 mL of the 2X stocks of compound are added to the cells.

After 24 hours, the media is aspirated from the assay plates. Each well is rinsed with Iml PBS, and the PBS is aspirated. 180PL SDS-PAGE sample buffer (Novex) containing 5% 2-mercaptoethanol is added to each well. The plates are heated to 100° C. for 5 minutes using a heat block containing an adapter for assay plates. The plates are placed on ice. After 10 minutes, 20μL of an RNAse/DNase mix is added per well. This mix is 1 mg/mL DNaseI (Worthington Enzymes), 0.25 mg/mL Rnase A (Worthington Enzymes), 0.5M Tris-HCl pH8.0 and 5OmM MgCl₂. The plate is left on ice for 10 minutes. Samples are then either loaded on the gel, or stored at −70° C. until use.

Each assay plate (usually 3 compounds, each in 4-point titrations, plus controls) requires one 15-well 14% Novex gel. 25 μl of each sample is loaded onto the gel. The gel is run at 15 mA for about 3.5 hours. It is important to run the gel far enough so that there will be adequate separation between 21 kd (Rap1) and 29 kd (Rab6).

The gels are then transferred to Novex pre-cut PVDF membranes for 1.5 hours at 30 V (constant voltage). Immediately after transferring, the membranes are blocked overnight in 20 ml Western blocking buffer (2% nonfat dry milk in Western wash buffer (PBS+0.1% Tween-20). If blocked over the weekend, 0.02% sodium azide is added. The membranes are blocked at 4° C. with slow rocking.

The blocking solution is discarded and 20ml fresh blocking solution containing the anti Rap1a antibody (Santa Cruz Biochemical SC1482) at 1:1000 (diluted in Western blocking buffer) and the anti Rab6 antibody (Santa Cruz Biochemical SC310) at 1:5000 (diluted in Western blocking buffer) are added. The membranes are incubated at room temperature for 1 hour with mild rocking. The blocking solution is then discarded and the membrane is washed 3 times with Western wash buffer for 15 minutes per wash. 20ml blocking solution containing 1:1000 (diluted in Western blocking buffer) each of two alkaline phosphatase conjugated antibodies (Alkaline phosphatase conjugated Anti-goat IgG and Alkaline phosphatase conjugated anti-rabbit IgG [Santa Cruz Biochemical]) is then added. The membrane is incubated for one hour and washed 3x as above.

About 2mL per gel of the Amersham ECF detection reagent is placed on an overhead transparency (ECF) and the PVDF membranes are placed face-down onto the detection reagent. This is incubated for one minute, then the membrane is placed onto a fresh transparency sheet.

The developed transparency sheet is scanned on a phosphorimager and the Rap1a Minimum Inhibitory Concentration is determined from the lowest concentration of compound that produces a detectable Rap1a Western signal. The Rap1a antibody used recognizes only unprenylated/unprocessed Rap1 a, so that the precence of a detectable Rap1a Western signal is indicative of inhibition of Rap1a prenylation.

Protocol C:

This protocol allows the determination of an EC₅₀ for inhibition of processing of Rap1a. The assay is run as described in Protocol B with the following modifications. 20 μl of sample is run on pre-cast 10-20% gradient acrylamide mini gels (Novex Inc.) at 15 mA/gel for 2.5-3 hours. Prenylated and unprenylated forms of Rap1a are detected by blotting with a polyclonal antibody (Rap1/Krev-1 Ab#121; Santa Cruz Research Products #sc-65), followed by an alkaline phosphatase-conjugated anti-rabbit IgG antibody. The percentage of unprenylated Rap1a relative to the total amount of Rap1a is determined by peak integration using Imagequant® software (Molecular Dynamics). Unprenylated Rapla is distinguished from prenylated protein by virtue of the greater apparent molecular weight of the prenylated protein. Dose-response curves and EC₅₀ values are generated using 4-parameter curve fits in SigmaPlot software.

### Example 17

In vivo tumor growth inhibition assay (nude mouse) In vivo efficacy as an inhibitor of the growth of cancer cells may be confirmed by several protocols well known in the art. Examples of such in vivo efficacy studies are described by N. E. Kohl et al. (Nature Medicine, 1:792-797 (1995)) and N. E. Kohl et al. (Proc. Nat. Acad. Sci. U.S.A., 91:9141-9145 (1994)).

Rodent fibroblasts transformed with oncogenically mutated human Ha-ras or Ki-ras (10⁶ cells/animal in 1 mL of DMEM salts) are injected subcutaneously into the left flank of 8-12 week old female nude mice (Harlan) on day 0. The mice in each oncogene group are randomly assigned to a vehicle, compound or combination treatment group. Animals are dosed subcutaneously starting on day 1 and daily for the duration of the experiment. Alternatively, the farnesyl-protein transferase inhibitor may be administered by a continuous infusion pump. Compound, compound combination or vehicle is delivered in a total volume of 0.1 mL. Tumors are excised and weighed when all of the vehicle-treated animals exhibited lesions of 0.5 -1.0 cm in diameter, typically 11-15 days after the cells were injected. The average weight of the tumors in each treatment group for each cell line is calculated.

What is claimed is:

1. A compound of formula A: 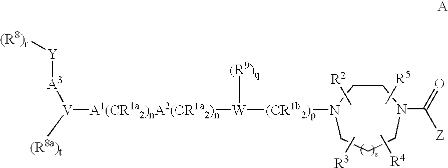
wherein:
R^{1a} and R^{1b} are independently selected from the group consisting of:
a) hydrogen,
b) aryl,
c) heterocyclyl,
d) C₃-C₁₀ cycloalkyl,
e) C₂-C₆ alkenyl,
f) C₂-C₆ alkynyl,
g) R¹⁰O—,
h) R¹¹S(O)ₘ—,
i) R¹⁰C(O)NR¹⁰—,
j) (R¹⁰)₂NC(O)—,
k) CN,
l) halo,
m) R¹⁰C(O)—,
n) R¹⁰OC(O)—,
o) —N(R¹⁰)₂,
p) R¹¹OC(O)NR¹⁰—, and
q) C₁-C₆ alkyl, said alkyl optionally substituted with aryl, heterocyclyl, C₃-C₁₀ cycloalkyl, C₂-C₆ alkenyl, C₂-C₆ alkynyl, R¹⁰O—, R¹¹S(O)ₘ—, R¹⁰C(O)NR¹⁰—, (R¹⁰)₂NC(O)—, CN, halo, R¹⁰C(O)—, R¹⁰OC(O)—, —N(R¹⁰)₂, or R¹¹OC(O)—NR¹⁰—;
R² and R³ are independently selected from the group consisting of:
a) H,
b) C₁₋₈ alkyl,
c) C₂₋₈ alkenyl,
d) C₂₋₈ alkynyl,
e) aryl,
f) heterocyclyl,
g) (C═O)NR⁶R⁷, and
h) (C═O)OR⁶,
said alkyl, alkenyl, alkynyl, aryl, and heterocyclyl optionally substituted with one or more substituents selected from the group consisting of:
1) aryl or heterocyclyl, unsubstituted or substituted with:
a) C₁₋₄ alkyl,
b) (CH₂)ₚOR⁶,
c) (CH₂)ₚNR⁶R⁷,
d) halo,
e) CN,
2) C₃₋₆ cycloalkyl,
3) OR⁶,
4) SOmR⁶a,
5) NR⁶R⁷,
6) NR⁶(C═O)R⁷,
7) NR⁶(C═O)NR⁷R^{7a},
8) —O(C═O)NR⁶R⁷,
9) O(C═O)OR⁶,
10) —(C═O)NR⁶R⁷,
11) —SO₂NR⁶R⁷,
12) NR⁶SO₂R^{6a},
13) —(C═O)R⁶,
14) —(C═O)OR⁶, and
15) halo; or
R² and R³ are attached to the same C atom and are combined to form —(CH₂)ᵤ— wherein one of the carbon atoms is optionally replaced by a moiety selected from: O, S(O)ₘ, —NC(O)—, and —N(COR¹⁰)—;
R⁴ and R⁵ are independently selected from H and C₁₋₄ alkyl;
R⁶, R⁷ and R^{7a} are independently selected from the group consisting of:
a) H,
b) C₁₋₈ alkyl,
c) C₃₋₆ cycloalkyl,
d) heterocyclyl,
e) aryl,
f) aroyl,
g) heteroaroyl,
h) arylsulfonyl, and
i) heteroarylsulfonyl,
said alkyl, cycloalkyl, heterocyclyl, aryl, aroyl, heteroaroyl, arylsulfonyl, and heteroarylsulfonyl is optionally substituted with one or more of the following:
1) C₁₋₄ alkoxy,
2) aryl,
3) heterocyclyl,
4) halo,
5) OH,
6) —(C═O)R¹¹,
7) —SO₂R¹¹,
8) C₁₋₄ alkyl, or
9) N(R¹⁰)₂;
R⁶ and R⁷ may be joined in a ring;
R⁷ and R^{7a} may be joined in a ring;
R^{6a} is selected from the group consisting of:
a) C₁₋₄ alkyl,
b) C₃₋₆ cycloalkyl,
c) heterocyclyl, and
d) aryl,
said alkyl, cycloalkyl, heterocyclyl, and aryl is optionally substituted with: one or more of the following:
1) C₁₋₄ alkoxy,
2) aryl,
3) heterocyclyl,
4) halogen,
5) OH,
6) —(C═O)R¹¹,
7) —SO2R¹¹,
8) C₁₋₄ alkyl, or
9) N(R¹⁰)₂;
R⁸ is selected from the group consisting of:
a) aryl,
b) heterocyclyl,
c) C₃-C₁₀ cycloalkyl,
d) C₂-C₆ alkenyl,
e) C₂-C₆ alkynyl,
f) C₁-C₆ perfluoroalkyl,
g) halo,
h) R¹⁰O—,
i) R¹¹ S(O)ₘ—,
j) R¹⁰C(O)NR¹⁰—,
k) (R¹⁰)₂NC(O)—,
l) CN,
m) R¹⁰C(O)—,
n) R¹⁰OC(O)—,
o) —N(R¹⁰)₂,
p) R¹¹OC(O)NR¹⁰—, and
q) C₁-C₆ alkyl, said alkyl is optionally substituted with aryl, cyanophenyl, heterocyclyl, C₃-C IO cycloalkyl, C₂-C₆ alkenyl, C₂-C₆ alkynyl, C₁-C₆ perfluoroalkyl, halo, R¹⁰O—, R¹¹S(O)ₘ—, R¹⁰C(O)N R¹⁰ —, (R¹⁰)₂NC(O)—, CN, R¹⁰C(O)—, R¹⁰OC(O)—, —N(R¹⁰)₂, or R¹¹ OC(O)NR¹⁰—;
R^{8a} is selected from the group consisting of:
a) aryl,
b) heterocyclyl,
c) C₃-C₁₀ cycloalkyl,
d) C₂-C₆ alkenyl,
e) C₂-C₆ alkynyl,
f) C₁-C₆ perfluoroalkyl,
g) halo,
h) R¹⁰O—,
i) R¹¹S(O)ₘ—,
j) R¹⁰C(O)NR¹⁰—,
k) (R¹⁰)₂NC(O)—,
l) CN,
m) R¹⁰C(O)—,
n) R¹⁰OC(O)-,
o) —N(R¹⁰)₂,
p) R¹¹OC(O)NR¹⁰—, and
q) C₁-C₆ alkyl unsubstituted or substituted by aryl, cyanophenyl, heterocycle, C₃-C₁₀ cycloalkyl, C₂-C₆ alkenyl, C₂-C₆ alkynyl, C₁-C₆ perfluoroalkyl, halo, R¹⁰O—, R¹¹S(O)ₘ—, R¹⁰C(O)NR¹⁰—, (R¹⁰)₂NC(O)—, CN, R¹⁰C(O)—, R¹⁰OC(O)—, —N(R¹⁰)₂, or R¹¹OC(O)NR¹⁰—;
R⁹ is selected from the group consisting of:
a) hydrogen,
b) C₂-C₆ alkenyl,
c) C₂-C₆ alkynyl,
d) C₁-C₆ perfluoroalkyl,
e) halo,
f) R¹⁰O—,
g) R¹¹S(O)ₘ—,
h) R¹⁰C(O)NR¹⁰—,
i) (R¹⁰)₂NC(O)—,
j) CN,
k) R¹⁰C(O)—,
l) R¹⁰OC(O)—,
m) —N(R¹⁰)₂,
n) R¹¹OC(O)NR¹⁰—, and
o) C₁-C₆ alkyl, said alkyl is optionally substituted with perfluoroalkyl, halo, R¹⁰O—, R¹¹S(O)ₘ—, R¹⁰C(O)NR¹⁰—, (R¹⁰)2NC(O)—, CN, R¹⁰C(O)—, R¹⁰OC(O)—, —N(R¹⁰)2, or R¹¹OC(O)NR¹⁰—;
R¹⁰ is hydrogen, C₁-C₈ alkyl, C₁-C₆ perfluoroalkyl, benzyl or aryl, said alkyl optionally substituted with OH or —OC₁-C₆ alkyl;
R¹¹ is C₁-C₆ alkyl or aryl;
A¹ and A² are independently selected from the group consisting of
a) a bond,
b) —CH═CH—,
c) —C≡C—,
d) —C(O)—,
e) —C(O)NR¹⁰—,
f) —NR¹⁰C(O)—,
g) —O—,
h) —N(R¹⁰)—,
i) —S(O)₂N(R¹⁰)—,
j) —N(R¹⁰)S(O)₂—, and
k) —S(O)ₘ—;
A³ is —C(O)—, —C(R^{1a})₂—, —O—, —N(R¹⁰)— or —S(O)ₘ—;
V is heteroaryl or aryl;
W is heterocyclyl;
Y is aryl;
Z is aryl or heterocyclyl,
said aryl and heterocyclyl is optionally substituted with one or more of the following:
1) C₁₋₈ alkyl, said alkyl optionally substituted with:
a) C₁₋₄ alkoxy,
b) NR⁶R⁷,
c) C₃₋₆ cycloalkyl,
d) aryl,
e) heterocyclyl,
f) OH,
g) —S(O)mR^{6a}, or
h) —C(O)NR⁶R⁷,
2) aryl,
3) heterocyclyl,
3) halo,
4) OR⁶,
5) NR⁶R⁷,
6) CN,
7) CF3,
9) —S(O)ₘR^{6a},
10) —C(O)NR⁶R⁷, and
11) C₃-C₆ cycloalkyl;
m is 0,1 or 2;
n is 0,1,2,3or 4;
p is 0, 1,2,3or 4;
q is 1 or 2;
r is 0, 1,2,3,4,or5;
s is 0 or 1;
t is 0, 1,2,3,4or5; and
u is 4or5;
or a pharmaceutically acceptable salt, stereoisomer or mixture thereof.

2. A compound of Formula B: 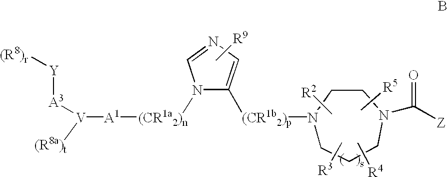
wherein:
R^{1a} and R^{1b} are independently hydrogen or C₁-C₆ alkyl, said alkyl optionally substituted with aryl, C₃-C₁₀ cycloalkyl, halo, R¹⁰O— or —N(R¹⁰)₂;
R², R³, R⁴ and R⁵ are independently selected from H and C₁₋₄ alkyl;
R⁶ and R⁷ are independently selected from the group consisting of:
a) H,
b) C₁₋₈ alkyl,
c) C₃₋₆ cycloalkyl,
d) aryl, and
e) heterocyclyl,
said alkyl, cycloalkyl, aryl, and heterocyclyl optionally substituted with:
1) C₁₋₄ alkoxy,
2) halo,
3) aryl,
4) heterocyclyl, or
5) C₁₋₄ alkyl;
R^{6a} is selected from:
a) C₁₋₄ alkyl,
b) C₃₋₆ cycloalkyl,
c) aryl, and
d) heterocyclyl,
said alkyl, cycloalkyl, aryl, and heterocyclyl optionally substituted with:
1) C₁₋₄ alkoxy,
2) halo,
3) aryl,
4) heterocyclyl, or
5) C₁₋₄ alkyl;
R⁸ is independently selected from the group consisting of:
a) aryl,
b) C₂-C₆ alkenyl,
c) C₂-C₆ alkynyl,
d) C₁-C₆ perfluoroalkyl,
e) halo,
f) R¹⁰O—,
g) R¹⁰C(O)NR¹⁰—,
h) CN,
i) R¹⁰C(O)—,
j) R¹⁰OC(O)—,
k) —N(R¹⁰)₂,
l) R¹¹OC(O)NR¹⁰—, and
m) C₁-C₆ alkyl, said alkyl is optionally substituted with C₁-C₆ perfluoroalkyl, R¹⁰O—, R¹⁰C(O)NR¹⁰—, R¹⁰C(O)—, R¹⁰OC(O)—, —N(R¹⁰)₂, or R¹¹OC(O)NR¹⁰—;
R8a is independently selected from the group consisting of:
a) aryl,
b) C₂-C₆ alkenyl,
c) C₂-C₆ alkynyl,
d) C₁-C₆ perfluoroalkyl,
e) halo,
f) R¹⁰O—,
g) R¹⁰C(O)NR¹⁰—,
h) CN,
i) R¹⁰C(O)—,
j) R¹⁰C(O)—,
k) —N(R¹⁰)₂,
l) R¹¹OC(O)NR¹⁰—, and
m) C₁-C₆ alkyl, said alkyl is optionally substituted with C₁-C₆ perfluoroalkyl, R¹⁰O—, R¹⁰C(O)NR¹⁰—, R¹⁰C(O)—, R¹⁰OC(O)—, —N(R¹⁰)₂, or R¹¹OC(O)NR¹⁰—;
R⁹ is selected from the group consisting of:
a) hydrogen,
b) halo,
c) R¹⁰O— and
d) C₁-C₆ alkyl;
R¹⁰ is hydrogen, C₁-C₈ alkyl, C₁-C₆ perfluoroalkyl, benzyl or aryl, said alkyl optionally substituted with OH or —OC₁-C₆ alkyl;
R¹¹ is C₁-C₆ alkyl or aryl;
A¹ is a bond, —CH═CH—, —C≡C—, —C(O)—, —C(O)NR¹⁰—, O, —N(R¹⁰)—, or —S(O)ₘ—;
A³ is —C(O)—, —C(R^{1a})₂—, O, —N(R¹⁰)— or S(O)ₘ;
V is:
a) heteroaryl, selected from the group consisting of imidazolyl, pyridinyl, thiazolyl, indolyl, quinolinyl, isoquinolinyl, and thienyl, or
b) aryl;
Y is aryl;
Z is aryl, said aryl optionally substituted with one or more of the following:
1) C₁₋₈ alkyl, unsubstituted or substituted with:
a) C₁₋₄ alkoxy,
b) NR⁶R⁷,
c) C₃₋₆ cycloalkyl,
d) aryl,
e) heterocyclyl,
f) OH,
g) —S(O)ₘR^{6a}, or
h) —C(O)NR⁶R⁷,
2) aryl,
3) heterocyclyl,
4) halo,
5) OR⁶,
6) NR⁶R⁷,
7) CN,
8) CF3,
9) —S(O)ₘR^{6a},
10) —C(O)NR⁶R⁷, or
11) C₃-C₆ cycloalkyl;
m is 0, 1 or 2;
n is 0,1,2,3 or 4;
p is 0,1, 2, 3 or4;
r is 0, 1,2,3,4,or5;
s is 0 or 1; and
t is 0 to 5;
or a pharmaceutically acceptable salt, stereoisomer, or mixture thereof.

3. A compound of formula C: 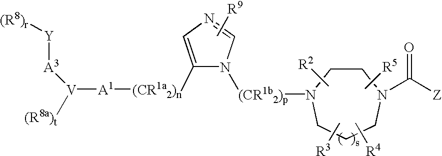
wherein:
R^{1a} and R^{1b} are independently hydrogen or C₁-C₆ alkyl, said alkyl optionally substituted with aryl, C₃-C₁₀ cycloalkyl, halo, R¹⁰O— or —N(R¹⁰)₂;
R², R³, R⁴ and R⁵ are independently selected from H and C₁₋₄ alkyl;
R⁶ and R⁷ are independently selected from the group consisting of:
a) H,
b) C₁₋₈ alkyl,
c) C₃₋₆ cycloalkyl,
d) aryl, and
e) heterocyclyl,
said alkyl, cycloalkyl, aryl, and heterocyclyl optionally substituted with:
1) C₁₋₄ alkoxy,
2) halo,
3) aryl,
4) heterocyclyl, or
5) C₁₋₄ alkyl;
R^{6a} is selected from:
a) C₁₋₄ alkyl,
b) C₃₋₆ cycloalkyl,
c) aryl, and
d) heterocyclyl,
said alkyl, cycloalkyl, aryl, and heterocyclyl optionally substituted with:
1) C₁₋₄ alkoxy,
2) halo,
3) aryl,
4) heterocyclyl, or
5) C₁₋₄ alkyl;
R⁸ is independently selected from the group consisting of:
a) aryl,
b) C₂-C₆ alkenyl,
c) C₂-C₆ alkynyl,
d) C₁-C₆ perfluoroalkyl,
e) halo,
f) R¹⁰O—,
g) R¹⁰C(O)NR¹⁰—,
h) CN,
i) R¹⁰C(O)—,
j) R¹⁰OC(O)—,
k) —N(R¹⁰)₂,
l) R¹¹OC(O)NR¹⁰—, and
m) C₁-C₆ alkyl, said alkyl is optionally substituted with C₁-C₆ perfluoroalkyl, R¹⁰O—, R¹⁰C(O)NR¹⁰—, R¹⁰C(O)—, R¹⁰OC(O)—, —N(R¹⁰)₂, or R¹¹OC(O)NR¹⁰—;
R^{8a} is independently selected from the group consisting of:
a) aryl,
b) C₁-C₆ alkyl,
c) C₂-C₆ alkenyl,
d) C₂-C₆ alkynyl,
e) C₁-C₆ perfluoroalkyl,
f) halo,
g) R¹⁰O—,
h) R¹⁰C(O)NR¹⁰—,
i) CN,
j) R¹⁰C(O)—,
k) R¹⁰OC(O)—,
l) —N(R¹⁰)₂,
m) R¹¹OC(O)NR¹⁰—, and
n) C₁-C₆ alkyl, said alkyl is optionally substituted with C₁-C₆ perfluoroalkyl, R¹⁰O—, R¹⁰C(O)NR¹⁰—, R¹⁰C(O)—, R¹⁰OC(O)—, —N(R¹⁰)₂, or R¹¹OC(O)NR¹⁰—;
R⁹ is selected from the group consisting of:
a) hydrogen,
b) halo,
c) R¹⁰O— and
d) C₁-C₆ alkyl;
R¹⁰ is hydrogen, C₁-C₈ alkyl, C₁-C₆ perfluoroalkyl, benzyl or aryl, said alkyl optionally substituted with OH or —OC₁-C₆ alkyl;
R¹¹ is C₁-C₆ alkyl or aryl;
A¹ is a bond, —CH═CH—, —C≡C—, —C(O)—, C(O)NR¹⁰—, O, —N(R¹⁰)—, or —S(O)ₘ—;
A³ is —C(O)—, —C(R^{1a})₂—, O, —N(R¹⁰)—or S(O)ₘ;
V is:
a) heteroaryl, selected from the group consisting of imidazolyl, pyridinyl, thiazolyl, indolyl, quinolinyl, isoquinolinyl, and thienyl, or
b) aryl;
Y is aryl;
Z is aryl, said aryl optionally substituted with one or more of the following:
1) C₁₋₈ alkyl, unsubstituted or substituted with:
a) C₁₋₄ alkoxy,
b) NR⁶R⁷,
c) C₃₋₆ cycloalkyl,
d) aryl,
e) heterocyclyl,
f) OH,
g) —S(O)ₘR^{6a}, or
h) —C(O)NR⁶R⁷,
2) aryl,
3) heterocyclyl,
4) halo,
5) OR⁶,
6) NR⁶R⁷,
7) CN,
8) CF_{3,}
9) —S(O)ₘR^{6a},
10) —C(O)NR⁶R⁷, or
11) C₃-C₆ cycloalkyl;
m is 0, 1 or 2;
n is 0, 1, or 2;
p is 0,1, or 2;
r is 1 to 3;
s is 1; and
t is 0 to 3;
or a pharmaceutically acceptable salt, stereoisomer, or mixture thereof.

4. A compound of Formula D 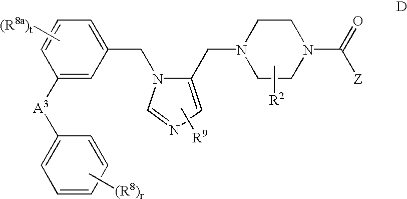
wherein
R² is H or C₁₋₄ alkyl;
R⁸ is CN, halo, C₁₋₆ alkyl, or CF₃;
R^{8a} is OR¹⁰, CN, halo, C₁₋₆ alkyl, or CF₃;
R⁹ is H or C₁₋₃ alkyl;
R¹⁰ is H, C₁₋₈ alkyl, C₁₋₆ perfluoroalkyl, benzyl, or aryl, said alkyl optionally substituted with OH or OC₁₋₈ alkyl;
A³ is O or S(O)ₘ;
Z is aryl, said aryl optionally substituted with one, two or three substituents selected from:
1) C₁₋₈ alkyl,
2) aryl,
3) heterocyclyl,
4) halo,
5) OH,
6) CN,
7) OC₁₋₆ alkyl, and
8) CF₃;
m is 0, 1, or 2; and
r and t are independently 0, 1, or 2.

5. The compound of claim 1 selected from the group consisting of:
1-(2-hydroxy-5 -methylbenzoyl)-4-[1-(3-((3-(2-hydroxyethoxy)phenyl)oxy)-4-cyanobenzyl)-2-methyl-5 -imidazolylmethyl]piperazine;
1-(2-methoxy-5-methylbenzoyl)-4-[1-(3 -((3-(2-hydroxyethoxy)phenyl)oxy)-4-cyanobenzyl)-2-methyl-5-imidazolylmethyl]piperazine;
1-(2-butoxy-5-methylbenzoyl)-4-[1-(3-((3-(2-hydroxyethoxy)phenyl)oxy)-4-cyanobenzyl)-2-methyl-5-imidazolylmethyl]piperazine;
1-(2-methoxybenzoyl)-4-[1-(3-((3-(2-hydroxyethoxy)phenyl)oxy)-4-cyanobenzyl)-2-methyl-5-imidazolylmethyl]piperazine;
1-(2-butoxybenzoyl)-4-[1-(3-((3-(2-hydroxyethoxy)phenyl)oxy)-4-cyanobenzyl)-2-methyl-5-imidazolylmethyl]piperazine;
1-(2-methoxy-4-methylbenzoyl)-4-[l1-(3-((3-(2-hydroxyethoxy)phenyl)oxy)-4-cyanobenzyl)-2-methyl-5 -imidazolylmethyl]piperazine;
1-(2-butoxy-4-methylbenzoyl)-4-[1 -(3 -((3-(2-hydroxyethoxy)phenyl)oxy)-4-cyanobenzyl)-2-methyl-5-imidazolylmethyl]piperazine;
1-(2-methoxy-3-methylbenzoyl)-4-[ 1 -(3-((3-(2-hydroxyethoxy)phenyl)oxy)-4-cyanobenzyl)-2-methyl-5-imidazolylmethyl]piperazine; and
1-(2-butoxy-3-methylbenzoyl)-4-[1-(3-((3-(2-hydroxyethoxy)phenyl)oxy)-4-cyanobenzyl)-2-methyl-5-imidazolylmethyl]piperazine;
or the pharmaceutically acceptable salts or optical isomers thereof.

6. The compound of claim 1 selected from the group consisting of 1-(2-butoxybenzoyl)-4-[1-(3-((3-(2-hydroxyethoxy)phenyl)oxy)-4-cyanobenzyl)-2-methyl-5-imidazolylmethyl]piperazine, 1-(2-methoxy-4-methylbenzoyl)-4-[1-(3-((3-(2-hydroxyethoxy)phenyl)oxy)-4-cyanobenzyl)-2-methyl-5-imidazolylmethyl] piperazine, and pharmaceutically acceptable salts or optical isomers thereof

7. 1-(tert-Butoxycarbonyl)-4-[1-(3-((3-(2-hydroxyethoxy)phenyl) oxy)-4-cyanobenzyl)-2-methyl-5-imidazolylmethyl]piperazine or a pharmaceutically acceptable salt or stereoisomer thereof.

8. A pharmaceutical composition comprising a pharmaceutical carrier and a compound of claim 1.

9. A method for inhibiting prenyl-protein transferase which comprises administering to a mammal in need thereof a therapeutically effective amount of a compound of claim 1.

10. A method for treating cancer which comprises administering to a mammal in need thereof a therapeutically effective amount of a compound of claim 1.

11. A method for treating neurofibromin benign proliferative disorder which comprises administering to a mammal in need thereof a therapeutically effective amount of a compound of claim 1.

12. A method for treating blindness related to retinal vascularization which comprises administering to a mammal in need thereof a therapeutically effective amount of a compound of claim 1.

13. A method for treating infections from hepatitis delta and related viruses which comprises administering to a mammal in need thereof a therapeutically effective amount of a compound of claim 1.

14. A method for preventing restenosis which comprises administering to a mammal in need thereof a therapeutically effective amount of a composition of a compound of claim 1.

15. A method for treating polycystic kidney disease which comprises administering to a mammal in need thereof a therapeutically effective amount of a compound of claim 1.

16. A method for treating or preventing a disease selected from cancer, neurofibromin benign proliferative disorder, blindness related to retinal vascularization, infections from hepatitis delta and related viruses, restenosis and polycystic kidney disease, which comprises administering to a mammal in need thereof a therapeutically effective amount of a compound of claim 1.

17. A pharmaceutical composition made by combining the compound of claim 1 and a pharmaceutically acceptable carrier.

18. A process for making a pharmaceutical composition which comprises combining a compound of claim 1 and a pharmaceutically acceptable carrier.

19. A method of conferring radiation sensitivity on a tumor cell which comprises administering to a mammal in need thereof a therapeutically effective amount of a compound of claim 1 in combination with radiation therapy.

20. A method for treating cancer which comprises administering to a mammal in need thereof a therapeutically effective amount of a compound of claim 1 in combination with an antineoplastic.

21. A method according to claim 20 wherein the antineoplastic is paclitaxel.
